(19) United States
(12) Patent Application Publication
(10) Pub. No.: US 2004/0143025 A1
(43) Pub. Date: Jul. 22, 2004

(54) Carbon monoxide generating compunds for treatment of vascular, inflammatory and immune disorders

(76) Inventors: Roland Buelow, Palo Alto, CA (US); Jacky Woo, San Jose, CA (US); Suhasini Iyer, San Ramon, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/661,462

(22) Filed: Sep. 13, 2003

Related U.S. Application Data
(63) Continuation-in-part of application No. 10/115,276, filed on Apr. 1, 2002.

(51) Int. Cl. A61K 31/02

(52) U.S. Cl. 514/758

(57) ABSTRACT

Methods and compositions are provided for treating vascular disease and modulating the inflammatory and immune processes using alkyl halides. The subject compounds are capable of inhibiting the proliferation of vascular smooth muscle cells, protecting the vasculature against oxidative stress and injury, modulating the activity of various immune system cells, inhibiting the production of pro-inflammatory cytokines and enhancing production of anti-inflammatory cytokines, thereby being effective in the treatment of conditions associated with adverse proliferative or inflammatory responses. Methods for extending the survival of an organ transplant and inhibiting chronic rejection in a recipient are also provided.

### STATEMENT OF RELATED APPLICATIONS

The present application claims priority to U.S. Provisional Application Serial No. 60/280,526, filed on Mar. 30, 2001 and is a continuation-in-part of U.S. application Ser. No. 10/115,276, filed Apr. 1, 2002, which applications are incorporated herein by reference.

### BACKGROUND OF THE INVENTION

The immune system is an extraordinarily complex combination of cells and compositions that protects a mammalian host against a wide variety of pathogens, while surveiling the body against deleterious aberrations, such as neoplasia. One branch of the immune system involves the cells that carry out immune system functions, including both (a) lymphocytes, such as the bone marrow-derived B-lymphocytes, the thymus-derived T lymphocytes and natural-killer (NK) cells, and (b) the mononuclear phagocytes, including both monocytes and macrophages. While lymphocytes are primarily associated with specific immune responses, due to their ability to specifically recognize and distinguish antigenic determinants, the mononuclear phagocytes are most often involved in the general removal of foreign microbes through phagocytosis as well as the production and secretion of cytokines as induced either directly by a microbe itself or in response to antigen-stimulated T lymphocytes. The functions of lymphocytic cells and the mononuclear phagocytes are highly interconnected and essential for proper immune system function.

Cytokines, such as the various interferons, interleukins, tumor necrosis factors, chemokines, hematopoietic growth factors and migration inhibition factors are a diverse group of proteins that are produced by a wide variety of different cells types of the immune system. Most importantly, cytokines are produced and/or responded to by various lymphocytes and mononuclear phagocytes in response to various stimuli. For the most part, cytokines are produced during the effector phases of both natural and specific immunity and serve to mediate and regulate both immune and inflammatory responses. Cytokines, like other polypeptide hormones, initiate their action by binding to specific receptors on the surface of target cells, their activation often resulting in an inflammatory response.

While activation of the immune response and cytokine-induced inflammatory responses are extremely important to a host's health and proper functioning of the immune system, there are a number of situations where such activation is undesired. One particular area is where a cytokine-mediated inflammatory response functions to adversely affect the health of the host, such as inflammatory responses associated with such maladies as septic shock, rheumatoid arthritis, Crohn's disease, colitis, and the like. Another incidence is where there is a failure on the part of CTLs in that they attack cells where the MHC and associated peptide are both endogenous, as occurs in autoimmune diseases such as insulin-dependent diabetes mellitus (IDDM). An additional incidence is associated with transplantation, where one rarely has an identical match between the donor and recipient of the MHC antigens.

Immunosuppression has become a general approach in situations where activation of CTLs is undesired. However, immunosuppressants such as cyclosporin A, FK506, and the like, have numerous undesirable side effects. Additionally, various approaches have been employed for controlling or inhibiting inflammatory responses, however, many of these approaches also have one or more undesirable effects. There is, therefore, substantial interest in identifying new agents which can act to inhibit the activation of lymphocytic cells, particularly CTLs, while having less of a universal immunosuppressive effect on the immune system and fewer side effects, so as to leave the host with a substantial proportion of the immune system for protection against adventitious infection. There is also a substantial interest in identifying new agents that function to control or inhibit adverse inflammatory reactions.

Heme oxygenases (HO) are the rate-limiting enzymes that catalyze the conversion of heme to biliverdin, carbon monoxide (CO) and free iron, the first step in the oxidative conversion of heme to bilirubin. HO-2 is the constitutive isoform present under physiological conditions, while HO-1 is the inducible isoform that provides protection against oxidative injury. Recently, great interest has been placed on the role of HO-1 in cellular responses to oxidative stress and insult, including ischemic and immunogenic effects. Upregulation or inducement of HO-1 expression has been found to produce a variety of potent anti-inflammatory and immunosuppressive effects, including prolongation of allograft survival and alleviation of graft versus host disease.

More recently, Otterbein et al., Nature America 6(4):422-28 (2000) have suggested that CO may mediate much or all of the anti-inflammatory effects seen with HO-1. Their data indicate that CO can selectively inhibit expression of the pro-inflammatory cytokines TNF-α, IL-1 β and MIP-1 β and may increase production of the anti-inflammatory cytokine IL-10. Subsequent data from these researchers suggests that the protective effect of HO-1 in preventing graft rejection may be mediated through the generation of CO. Thus, there is substantial interest in developing CO-based approaches to treating different manifestations of inflammatory diseases and for improving transplant outcome, including chronic rejection, where a drug may act by itself or in conjunction with other drugs.

The heme oxygenase pathway also plays a critical role in regulating and maintaining vascular tone to ensure adequate tissue oxygenation and perfusion. Vascular cells respond to an environment of oxidative stress by inducing endogenous antioxidant defense mechanisms. The main intracellular regulator under physiologic conditions is endothelial-derived nitric oxide (NO), which maintains normal vascular tone through its regulation of cyclic guanosine 3′,5′-monophosphate (cGMP) levels in vascular smooth muscle cells (VSMC) by guanylate cyclase activation. In situations where NO production is impaired, such as hypoxia or atherogenesis, induction of heme oxygenase may provide an important secondary line of antioxidant defense through generation of the antioxidant bilirubin and the vasodilator CO.

Recent reports have suggested that VSMC-derived CO may take over as the regulator of gene expression and cGMP levels in vascular endothelial and smooth muscle cells in such situations. Morita et al., J. Clin. Investigation 96:2676-2682 (1995); Siow et al., Cardivascular Res. 41:385-394 (1999). In particular, CO has been identified as a dilator of VSMC via a cGMP-mechanism, and has been shown to suppress endothelin-1 (ET-1) and platelet-derived growth factor-B gene expression in endothelial cells and subsequently inhibit the proliferation of smooth muscle cells. CO also has endothelial cell-independent effects on VSMC proliferation through its suppression of E2F-1 gene expression, a transcription factor implicated in the control of cell cycle progression. Morita et al., J. Biol. Chem. 272(52):32804-9 (1997).

Thus, endogenous CO generated by the heme oxygenase pathway also protects against excessive VSMC proliferation, a main event in the pathogenesis of many cardiovascular diseases including atherosclerosis, intimal hyperplasia and pulmonary hypertension. VSMC proliferation and accumulation is also implicated in neointimal development elicited by arterial injury, such as denudation caused by balloon injury. Togane et al., Am J. Physiol. Heart Circ. Physiol. 278:H623-H632 (2000). Balloon injury induces the production of several vasoactive factors, including ET-1, and exposes the VSMC layer directly to red blood cells in the blood stream, which may change the shear stress and redox state in the vascular wall. CO inhibits neointimal formation and thus serves a critical protective function for arterial injury as well.

Unfortunately, however, there is presently lacking a practical and predictable therapeutic modality for increasing cellular carboxyhemoglobin levels. Given the toxicities associated with prolonged inhalation of exogenous CO, there is a pressing need to find alternative modalities useful for modulating carboxyhemoglobin levels both systemically and locally, as necessary for prophylactic and therapeutic treatment of inflammatory, immune and vascular diseases. These modalities may find use in conjunction with other drugs, where lower levels of other drugs having significant side effects may be used effectively, so as to reduce the detrimental side effects. There is also a substantial interest in developing new approaches to reducing the risk of atherosclerosis, and minimizing complications associated with surgical procedures that cause injury to arterial walls, such as balloon angioplasty. The present invention addresses and resolves all of these concerns.

### BRIEF DESCRIPTION OF THE RELEVANT LITERATURE

Heme oxygenase has been the subject of numerous studies as evidenced by the review article, Abraham et al., Int. J. Biochem. 20(6):543-558 (1988), and by Raju and Maines, Biochimica et Biophysica Acta 1217:273-280 (1994); Neil et al., J of Ocular Pharmacology and Therapeutics 11(3):455-468 (1995); Haga et al., ibid. 1316:29-34 (1996); Willis et al., Nature Medicine 2(1):87-90 (1996); and Agarwal et al., Transplantation 61(1):93-98 (1996).

Modulation of heme oxygenase activity has been described in U.S. Pat. Nos. 5,756,492 & 6,060,467 and in International PCT Publication No. WO 00/36113, the disclosures of which are incorporated by reference herein, as well as in Woo et al., Transplantation 69(4):623 (2000); DeBruyne et al., Transplantation 69(1):120 (2000); Amersi et al., J. Clin. Invest. 104(11):1631-39 (1999); Cuturi et al.; Mol. Med. 5(12):820 (1999); Brouard et al., Transplantation 67(12):1614-31 (1999); Hancock et al., Nature Med. 4(12):1392-96 (1998); Squiers et al., Transplantation 66:1558-65 (1998); Woo et al., Transplant. Immunol. 6(2):84-94 (1998); and Iyer et al., J. Biol. Chem. 273(5):2692-97.

More recently, Otterbein et al. have suggested that carbon monoxide has anti-inflammatory effects involving the mitogen-activated protein kinase pathway. Nature America 6(4):422-428 (2000); while Sato and colleagues have demonstrated that exogenous CO can substitute for heme oxygenase in preventing graft rejection; J. Immunol. 166:4185-94 (2001); and Brouard and colleagues have demonstrated that CO suppresses endothelial cell apoptosis. J. Exp. Med. 192(7):1015-25 (2001).

With respect to the induction of heme oxygenase in vascular diseases, Siow et al., supra, reviews the role of heme oxygenase, CO and bilirubin in atherogenesis. Togane et al. report on the protective roles of endogenous CO in neointimal development elicited by arterial injury, supra, while Duckers et al. suggest that the anti-proliferative effects of HO-1 may be protective under conditions of vascular injury even in the absence of hypoxia. Nature Med. 7:693-698 (2001).

### SUMMARY OF THE INVENTION

The present invention provides methods and compositions for treating vascular, inflammatory and immune diseases using carbon monoxide generating compounds, which are capable of being metabolized into carbon monoxide in vivo. Preferred carbon monoxide generating compounds for use in the subject invention include alkyl halides, e.g., haloalkanes or haloalkenes generally having from one to four carbon atoms and two or more halogen substitutions.

In a preferred embodiment, the alkyl halide is a dihalomethane. In a particularly preferred embodiment, the dihalomethane is methylene chloride (CH₂Cl₂), which is metabolized in vivo into CO and CO₂.

In an alternative and more preferred embodiment, the alkyl halide is a trihalomethane or haloform. In a particularly preferred embodiment, the haloform is iodoform (CHI₃) and/or bromoform (CHBr₃). As demonstrated herein, iodoform and bromoform provide equal or better carboxyhemoglobin production and therapeutic efficacy using lower amounts of compound, in comparison with MC.

In another aspect, the invention provides a pharmaceutical composition for the treatment of vascular, inflammatory and immune disorders in a mammal, comprising a carbon monoxide generating compound capable of increasing the carboxyhemoglobin level in said mammal. Preferably, the carbon monoxide generating compound is an alkyl halide, and still more preferably, a dihalomethane and/or a haloform, which may be administered alone or in a pharmaceutically acceptable vehicle. Also provided is a method for increasing the carboxyhemoglobin level in a mammal, comprising the administration of an alkyl halide to said mammal in an amount sufficient to increase the blood carboxyhemoglobin level to between about 1 and 10%, more preferably between about 2 and 9%, still more preferably between about 4 and 6%, yet more preferably between about 3 and 8%, and generally between about 3 and 10%.

In a further aspect, the present invention provides methods and compositions for modulating inflammatory and immune processes throughout the body using alkyl halides. The subject compounds are capable of modulating the activity of various immune system cells, inhibiting the production of pro-inflammatory cytokines and enhancing production of anti-inflammatory cytokines by cells capable of producing such cytokines, thereby being effective in the treatment of conditions associated with adverse inflammatory responses.

Methods for extending the survival of an organ transplant in a recipient are also provided, wherein those methods comprise administering to said recipient an alkyl halide that functions to modulate the immune response against the transplanted organ, whereby the survival time of the organ transplant in the recipient is extended. Administration of the alkyl halides may be ex vivo of an organ to be transplanted or in vivo by any convenient means, including parenteral, systemic or localized administration, in sufficient amount to substantially inhibit lymphocyte activation and the inflammatory process through modulation of anti- and pro-inflammatory cytokine production.

In the vasculature, the subject compounds are capable of regulating vascular tone, inhibiting VSMC proliferation and protecting against oxidative stress and hypoxia, which have profound effects on vascular tone, endothelial permeability and coagulating function. Thus alkyl halides will also find use in treating vascular proliferative diseases and other disorders associated with HO-1 induction in response to oxidative stress.

In one embodiment, methods for inhibiting neointimal formation and improving the outcome of invasive vascular procedures are provided, comprising administering to a patient undergoing a procedure requiring or involving arterial injury such as balloon angioplasty at least one alkyl halide that functions to protect against neointimal development. In another embodiment, alkyl halides are employed to prevent atherogenesis, either in response to a specific oxidative event in the vasculature or prophylactically in patients at higher risk, such as, e.g., those with high levels of low-density lipoproteins (LDL) thought to be involved in atherogenesis.

Preferred alkyl halides for use in the subject methods include, e.g., haloalkanes and/or haloalkenes generally having from one to four carbon atoms and two or more halogen substitutions, and still more preferably, dihalomethanes and/or haloforms. Most preferred are iodoform, bromoform and/or methylene chloride. Administration of the alkyl halides according to the present invention may be by any convenient means, including parenteral, systemic or localized administration, in sufficient amount to substantially inhibit VSMC proliferation and modulate the vascular response to oxidative stress.

Additional embodiments will become evident upon a reading of the present specification.

### BRIEF DESCRIPTION OF THE DRAWINGS

FIGS. 1A, 1B & 1C are graphs of the levels of serum TNF-α, carboxyhemoglobin and O₂Hb in mice treated with LPS with or without 500 ppm gaseous CO.

FIG. 2 is a graph of the effect of methylene chloride administration on LPS-induced TNF-α production.

FIG. 3 is a graph of the effect of methylene chloride administration on blood carboxyhemoglobin levels.

FIG. 4 is a graph of the effect of exogenous CO on portal vein resistance in an ex vivo rat liver model of cold ischemia followed by reperfusion.

FIG. 5 is a graph showing the effect of exogenous CO on bile production in an ex vivo rat liver model of cold ischemia followed by reperfusion.

FIG. 6 is a graph showing the effect of exogenous CO on neutrophil activity as measured by a myeloperoxidase assay in an ex vivo rat liver model of cold ischemia followed by reperfusion.

FIG. 7 is a graph showing the effect of exogenous CO on COHb levels in an ex vivo rat liver model of cold ischemia followed by reperfusion.

FIG. 8 is a graph showing is a graph showing the effect of exogenous CO on bile production in an ex vivo rat liver model of cold ischemia followed by reperfusion, with and without the addition of L-NAME (an inducible NO inhibitor) or LY-83583 (a cGMP analogue) or pretreatment with ZnPP, an HO-1 inhibitor.

FIG. 9 is a graph showing the effect of exogenous CO on bile production in an ex vivo rat liver model of cold ischemia followed by reperfusion, with and without the addition of SB203580, a p38 MAPK inhibitor.

FIG. 10 is a graph showing in vitro cytotoxicity to Fas-bearing YAC-1 target cells after exposure to Yac-1 and Hela cells transfected with Ad-CD95+Ad-HO-1 (filled bars) and AD-CD95+Ad-β-gal (open bars).

FIG. 11 is a graph showing a pharmacokinetic study of systemic carboxyhemoglobin (COHb) levels after oral methylene chloride administration in a rat aorta model.

FIG. 12 is a chart showing computer-assisted morphometry of intima thickness in syngeneic and allogeneic rat aortic grafts at day 30 after transplantation, when treated with control (Add1324), Ad-HO-1 or methylene chloride.

FIG. 13 is a graph illustrating alloantibody levels in recipients of aortic allografts treated with AdHO-1 or CO.

FIG. 14 is a chart showing the arthritic score in control and methylene chloride-treated rats in a rat collagen-arthritis model.

FIG. 15 is a chart comparing the arthritic score obtained in rats treated with methylene chloride (RB2000), iodoform (RB2003) and bromoform (RB2002) in a rat collagen-arthritis model.

FIGS. 16A, B & C are graphs demonstrating the induction of the inflammatory cytokines TNF-α, IFN-γ and IL-6 in animals treated with methylene chloride (RB2000), iodoform (RB2003) and bromoform (RB2002) in comparison with placebo.

FIG. 17 is a chart comparing the arthritic score obtained in rats treated with varying amounts of iodoform (RB2003) in a rat collagen-arthritis model.

FIG. 18 is a chart showing the clinical score of animals in an EAE model of multiple sclerosis using methylene chloride and iodoform.

FIG. 19 is a graph showing effect of alkyl halide administration on blood carboxyhemoglobin levels.

### DESCRIPTION OF THE SPECIFIC EMBODIMENTS

Methods and compositions are herein provided for treating vascular, inflammatory and immune diseases through the use of carbon monoxide generating compounds in vitro and in vivo. As indicated herein, the subject compounds are capable of mediating the cytoprotective activity of HO-1, both in vitro and in vivo. Therefore, the subject compounds may be used in situations where one wants to mimic the anti-inflammatory and other protective effects seen with upregulation of HO-1. Preferred carbon monoxide generating compounds include at least one alkyl halide, e.g., haloalkanes and/or haloalkenes generally having from one to four carbon atoms and at least two halogen substitutions.

In one aspect of the present invention, the alkyl halides find use for regulating vascular tone, inhibiting VSMC proliferation and protecting against oxidative stress, thereby being useful for treating various disorders such as atherogenesis, restenosis, pressure or volume overload of the heart, hypertension, subarachnoidal hemorrhage, neointima formation and development, vasoconstriction, edema in the lung, and thrombus formation in the venous circulation.

In one such embodiment, methods for inhibiting neointimal formation and improving the outcome of invasive vascular procedures are provided, comprising administering to a patient undergoing a procedure involving arterial injury such as balloon angioplasty at least one alkyl halide that functions to protect against neointimal development. In another embodiment, the alkyl ahlides are employed to prevent atherogenesis, either in response to a specific oxidative event or prophylactically in patients at higher risk, such as, e.g., those having high levels of low-density lipoproteins (LDL) thought to be involved in atherogensis.

Another aspect provides methods and compositions for modulating inflammatory and immune processes in vitro and in vivo. The subject alkyl halides find use for inhibiting the production of inflammatory cytokines and enhancing the production of anti-inflammatory cytokines, including TNFα, interferons such as interferon-γ, interleukins such as IL-1, IL-4, IL-5, IL-6, IL-8, IL-10, IL-12, IL-13, IL-16, MIP 1α, chemokines, hematopoietic growth factors and the like, thereby being useful for inhibiting inflammatory responses associated with various disorders such as rheumatoid arthritis, septic shock, Crohn's disease, colitis, multiple sclerosis, granulomatous inflammation, hepatitis, allergic reactions, autoimmune diseases, ischemic/reperfusion injury, and the like, and delaying the onset of IDDM in a patient at risk for developing IDDM, both in vitro and in vivo. In a particularly preferred embodiment, the alkyl halides find use in treating rheumatoid arthritis and multiple sclerosis, improving the outcome of organ transplantation (e.g, kidney, liver, heart, etc.) and preventing ischemia/reperfusion injury.

The above-described carbon monoxide generating compounds will function both in vivo and in vitro to modulate inflammation and/or the immune response in a host or sample, respectively, into which they are introduced. The modulation will generally be exemplified by an inhibition of the expression of pro-inflammatory cytokines and/or an increase in the production of anti-inflammatory cytokines. Reliable and sensitive assays for determining the expression levels of such cytokines are well known and commercially available from such sources as BioSource International, Inc. in Camarillo, Calif.

By “carbon monoxide generating compounds” is meant compounds capable of metabolic conversion into carbon monoxide and other biocompatible breakdown products. As described and exemplified herein, suitable carbon monoxide generating compounds for use in the subject invention include alkyl halides, and more preferably, haloalkanes, haloalkenes and/or haloforms, generally having from one to four carbon atoms and substituted with at least two halogens.

In a preferred embodiment, the alkyl halide is a halomethane, and still more preferably, methylene chloride (MC), which is metabolized exclusively into CO and CO₂ via the cytochrome P-450 oxidative system. Gargas et al., Toxicol. Appl. Pharmacol. 87:211-23 (1986); Andersen et al., Toxicol. Appl. Pharmacol. 87:185-205 (1987). Carbon monoxide generated by the metabolism of the subject compounds, e.g., methylene chloride, will bind in vivo to hemoglobin so as to increase the patient's carboxyhemoglobin (COHb) level to a therapeutic range.

In an alternative and more preferred embodiment, the alkyl halide is a trihalomethane (haloform), with iodoform and bromoform most preferred. Haloforms are also metabolized into CO and CO₂ via a cytochrome P-450-dependent mixed function oxidase system, with in vivo metabolism following the halide order. Ahmed et al., Drug Metab. Dispos. 5(2):198-204 (1977); Anders et al., Drug Metab. Dispos., 6(5):556-60 (1978). Iodoform is particularly preferred for use as a carbon-monoxide generating compound in the subject methods in view of the superior efficacy obtained using reduced amounts of active compound, as demonstrated herein. Preferably, the alkyl halide is administered to a patient in an amount sufficient to increase the patient's systemic (i.e., blood) COHb level to about 1-10%, more preferably 2-9%, still more preferably 6-9%, most preferably 3-8%, usually 3-10%. Monitoring of the resulting COHb levels may be readily accomplished using sensitive assays known and available to the skilled artisan, for systemic monitoring as well as for monitoring in individual tissues or organs. See, e.g., Wong et al., Trans. Am. Clin. Climatol. Assoc. 111 (1):61-75 (2000).

The subject carbon monoxide generating compounds may be formulated in a variety of ways, depending upon the nature and purpose of administration, the specific inflammatory disease being treated, the particular generating compound, the number of administrations, the inclusion or use of other drugs, and the like, and such may be determined empirically by those skilled in the art. The formulation will generally be in a physiologically acceptable form, and may include various carriers or solvents such as water, deionized water, phosphate buffered saline, aqueous ethanol, glucose, propylene glycol, vegetable oils, olive oil or the like. In some instances, the subject carbon monoxide generating compounds may be formulated in a slow release formulation, where the subject compounds may be encapsulated in a wide variety of carriers, may be administered as capsules, or as a prodrug. The formulations may also include bacterial agents, stabilizers, buffers, or the like.

The subject carbon monoxide generators may also find use in adjunctive therapy with other antiinflammatory compounds (e.g., steroids, non-steroidal antiinflammatory agents (NSAIDS), monoclonal antibodies such as Remicade®, cytokine antagonists or inhibitors such as Enbrel® (TNF inhibitor), and the like) or immunosuppressive drugs (e.g., cyclosporine, Prograf® (FK-506), mycophenolate, monoclonal antibodies such as Simulect®, Zenapax®, or other biologics such as Thymoglobulin®, Lymphoglobuline®, and the like), where reduced amounts of the drug may be used, generally reducing the amount employed by at least 25%, more usually at least 40% or more, from the therapeutic dosage for the indication. The subject compounds may also be advantageously combined with other agents that may be employed in the treatment of the specific disease indications discussed herein (e.g. antibiotics, anti-metabolites or other cytotoxic agents, human leukocyte antigens, cyclooxygenase inhibitors, lipid-altering agents, ACE inhibitors or other vasodilators, sulfasalazine and related compounds, and the like).

The subject generator compounds may be administered either in vivo, ex vivo or in vitro, and may be taken parenterally or orally, generally being administered intravascularly, subcutaneously, intravenously or intramuscularly. In vivo delivery also includes, but is not limited to, direct injection via catheter or by other means of perfusion into a vessel, organ or tissue involved in or affected by an adverse proliferative, inflammatory or immune response. The subject compounds may be administered intravascularly at a location proximal to a transplanted organ or inflamed tissue, for example, or administered systemically. One of ordinary skill in the art will recognize the advantages and disadvantages of each mode of delivery, and will be able to determine a satisfactory means of delivery and delivery regimen without undue experimentation. For the subject alkyl halides, oral administration is most preferred.

The amount administered will vary depending upon what is being administered, the purpose of the administration, such as prophylaxis or therapy, the state of the host, the manner of administration, the number of administrations and the interval between administrations, and the like, all of which may be determined empirically by those skilled in the art. Applying these factors, the dosage will generally be in the range of about 5-500 mg/kg, usually in the range of about 25-500 mg/kg, more usually in the range of about or 100-500 mg/kg, most preferably in the range of 250-500 mg/kg. When administered parenterally, the total amount of the subject carbon monoxide generating compound per day will generally be in the range of about 1-500 mg/kg, more usually in the range of about 1-100 mg/kg, most preferably in the range of 1-10 mg/kg.

The dose may be in a single bolus or may be divided up and administered in portions to provide the desired level of carbon monoxide in the host over a period of time, and will be adjusted based on the metabolic conversion rate of the subject compound. With methylene chloride, for example, only about 50-80% of the compound is converted into carbon monoxide. Thus, administration of 10-500 mg/kg methylene chloride will typically result in about 3-165 mg/kg CO in vivo. Information relating to the pharmacokinetics and metabolism of such compounds is known in the art and available to the skilled artisan for empirically determining the proper dosages. See, e.g., Angelo et al., J Pharmacokinetics and Biopharmaceutics 12(4):413-435 (1984); Ahmed et al., Drug Metab. Dispos. 5(2):198-204 (1977); Anders et al., Drug Metab. Dispos., 6(5):556-60 (1978).

Methylene chloride is a preferred embodiment herein in that it has a near linear dose-response relationship, thus providing the skilled artisan with control over the degree of COHb formation so as to maintain COHb levels within the desired therapeutic range. Thus, MC provides a considerable advantage over other therapeutic modalities in that the predictability of its dose-response relationship enables maintenance of a therapeutic level of COHb while avoiding the toxicities associated with severe CO poisoning, e.g., carboxyhemglobinemia.

Even more preferred are haloforms, and iodoform and bromoform in particular, in view of the enhanced COHb production obtained with their parenteral use, using lower amounts of active compound in comparison with methylene chloride. Thus, these alternative preferred embodiments offer the same advantages as methylene chloride and further provide improved efficacy while at the same time reducing the amount of compound which must be administered to achieve therapeutic effect. Additionally, they provide the clinical benefit of resolving the carcinogenic risks associated with high levels of methylene chloride exposure.

In humans, the carbon monoxide generating compound will preferably be orally administered in an amount between about 1-100 mg/kg, more preferably between about 1-80 mg/kg, most preferably between about 1-60 mg/kg, generally between about 1-30 mg/kg.

As indicated above, the carbon monoxide generating compounds described herein also find use for inhibiting the activation of immune system cells, either by themselves or in conjunction with other immunosuppressant agents, particularly in extending the lifetime of transplants. In an alternative embodiment, therefore, the present invention provides a method for prolonging the acceptance of transplants in a mammalian host, which employs the administration of a carbon monoxide generating prior to, concomitant with, subsequent to or a combination thereof with the transplant. A particular regimen is employed for administration, where a single bolus or plurality of doses may be administered to the recipient and/or donor before, concomitant with, or subsequent to the implanting of the organ in the recipient. The particular protocol will depend upon the nature of the organ, whether the donor, recipient or organ is being treated, the particular carbon monoxide generating compound which is employed, and the use of other immunosuppressants.

Administration may begin within 14 days prior to the transplant, preferably within about 3 days, and desirably will include the day prior to the transplant and most preferably, the same day as and/or the day after the transplantion. Administration may be on consecutive days or non-consecutive days, generally any gap fewer than 10 days. In a preferred embodiment, administration concomitant with the transplant or on the same day is employed, and in a particularly preferred embodiment administration will begin on the same day as the transplant or the day before, and may be continued until the transplant is stabilized, generally not exceeding twelve months, more usually not exceeding four to twelve weeks. However, after implantation, the subject compounds may be administered as needed, depending upon the response of the recipient to the organ or cells. In some situations, the subject compounds may be administered chronically, as long as the implant is present in the host. The carbon monoxide generating compound may also be administered to the donor, usually within three days of the removal of the organ, more usually not later than the day prior to removal of the organ, desirably within about 12 hours of the removal of the organ.

The subject carbon monoxide generating compounds may be used with a wide variety of hosts, particularly primates, more particularly humans, or with domestic animals, and the like. The subject carbon monoxide generating compositions may be used in conjunction with the transplantation of a wide variety of organs, such as kidney, heart, liver, spleen, bone marrow, pancreas, lung, islet of langerhans, etc.

Generally, the graft life will be extended for at least three days beyond what could normally be anticipated in the absence of the subject carbon monoxide compounds, more usually at least five days. This can be useful in areas where xenogeneic grafts have been used awaiting an allogeneic graft, to allow for reduced amounts of immunosuppressants or avoid using immunosuppressants altogether. The subject compounds may be used for allogeneic, as well as xenogeneic, grafts.

### EXPERIMENTAL

The following examples are offered by illustration and not by way of limitation.

### Example 1

### Exogenous CO Administration

To examine the effect of gaseous CO on the immune system, C57/BL6 mice (B6, Jackson Laboratory, Bar Harbor, Me.) were first exposed to 500 ppm CO in air (Praxair, Danbury, Conn.) for one hour in a sealed chamber before injection of lipopolysaccharide (LPS) (0.3 mg/kg, i.v., Sigma, St Louis, Mo.). After injection, they were exposed to another hour in the CO chamber. Blood samples were collected one hour after LPS injection (through the aortic artery) and the COHb level in whole blood was measured by a whole blood AVOXimeter 4000 (A-VOX Systems, San Antonio, Tex.). Serum samples were separated and were kept at −80° C. until analysis. Serum TNF-α was measured by sandwich ELISA (Biosource, Camarillo, Calif.).

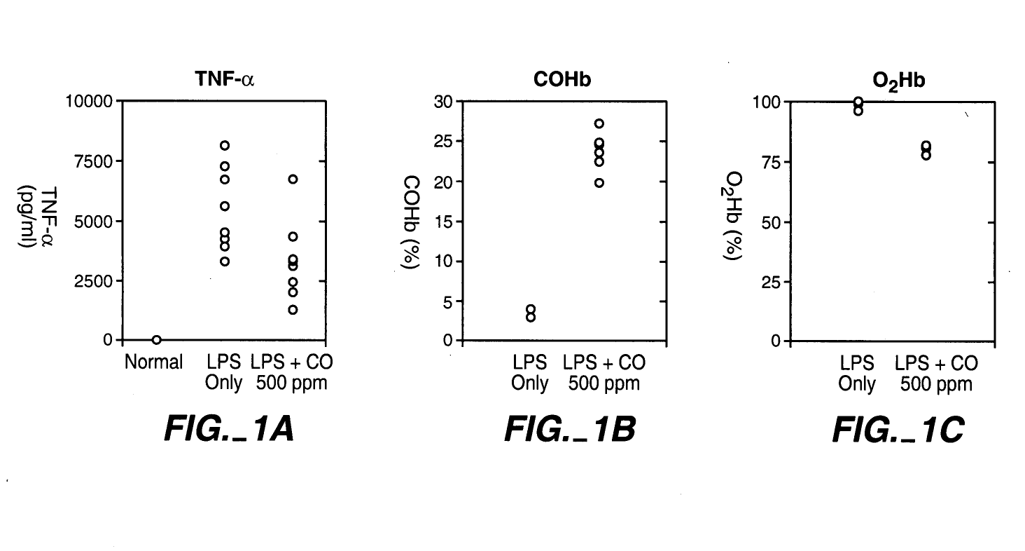

The results of this experiment are shown in FIGS. 1A-1C. Mice treated with LPS at 0.3 mg/kg alone produced a high level of TNF-α (5090.7±1595 pg/ml). Mice that were exposed to gaseous CO at 500 ppm showed a significant reduction (p<0.05) in serum TNF-α levels (3,347+1393 pg/ml) (FIG. 1A). COHb levels in the treated mice were also measured. As expected, mice exposed to gaseous CO had a significantly higher COHb percentage (23.83±2.48% p<0.01) compared to mice that were exposed to air (3.65+0.43%) (FIG. 1B). Concomitantly, the increase in COHb levels in mice exposed to gaseous CO was associated with a reduction in O₂Hb levels (FIG. 1C, 79.18±1.569% in CO-treated mice and 97.53±1.67% in non-treated mice respectively).

### Example 2

### Methylene Chloride as a Carbon Monoxide Generating Compound

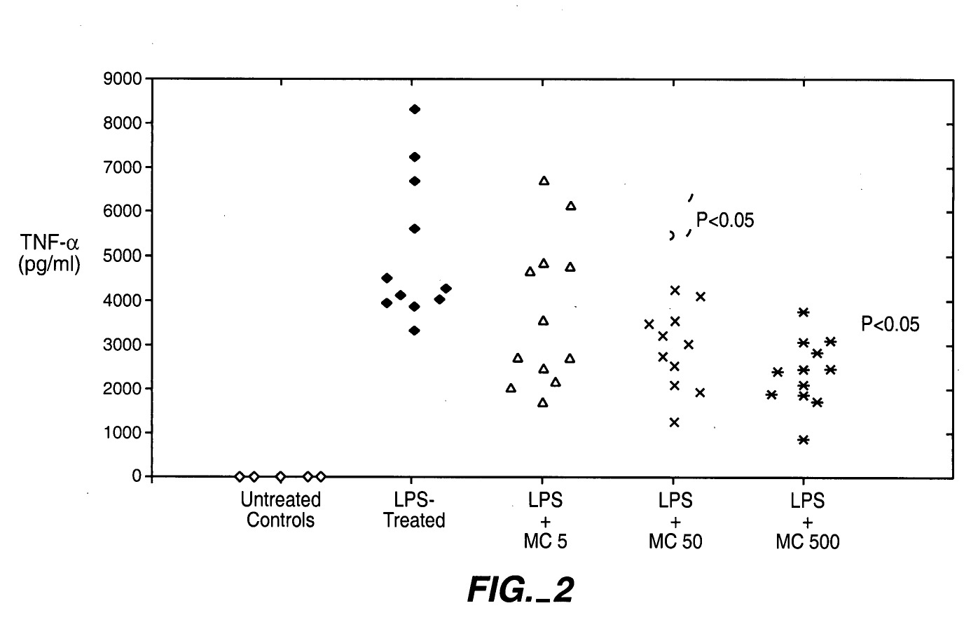

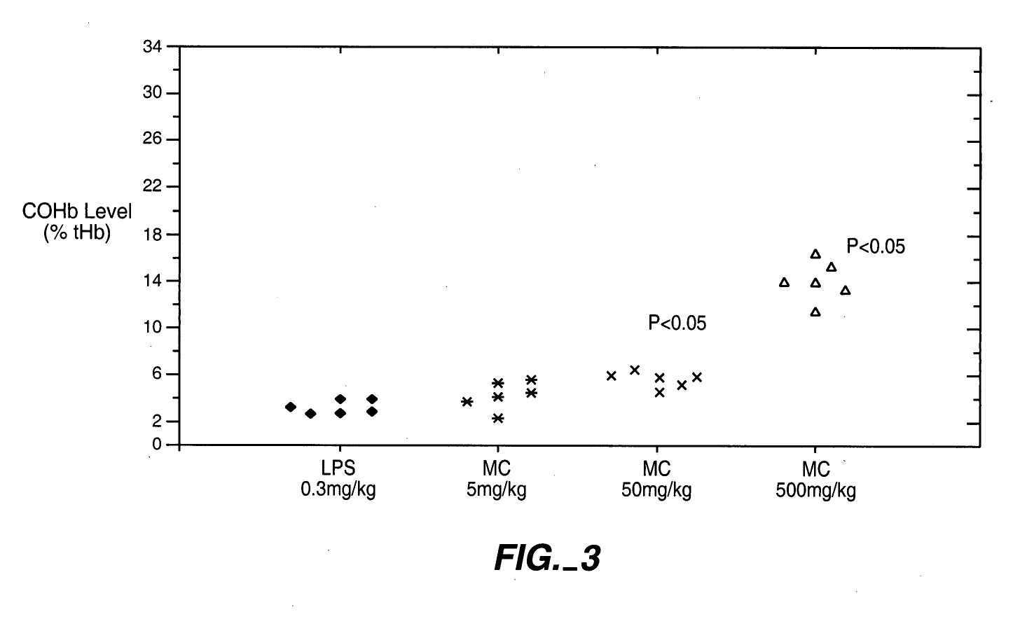

To reveal the therapeutic potential of CO generators, methylene chloride (MC, Sigma, St. Louis, Mo.) was selected as a lead compound. Different concentrations of MC were prepared by using olive oil as a solvent. Mice were treated with MC at 5 mg/kg., 50 mg/kg, and 500 mg/kg, p.o., one hour before LPS administration. As shown in FIG. 2, while there is a small and insignificant difference (p=0.06) in the level of TNF-α from mice treated with MC at 5 mg/kg (3720±1666 pg/ml) compared to LPS-treated controls (5090.7±1595 pg/ml), mice treated with MC at 50 mg/kg and 500 mg/kg had a significant reduction in TNF-α levels (3124.2±1147 pg/ml, p<0.05 and 2339±770 pg/ml, p<0.05, respectively). The dose-dependent reduction in TNF-αt was associated with a dose-dependent change in COHb. Mice treated with MC 5 mg/kg had no significant difference in COHb levels (4.22±1.2%) compared to mice without MC treatments (3.1±0.6%) (FIG. 3). However, mice that were treated with MC at 50 mg/kg and MC at 500 mg/kg had a significant increase in COHb levels (5.48±0.7% COHb, p<0.05 and 13.92±1.7% COHb, p<0.05) compared to untreated mice.

It should be noted that mice treated with gaseous CO had a higher level of COHb than mice treated with the inhibitory dosages of MC. This may be due to the fact that the majority of the inhaled CO is captured by pulmonary hemoglobin rather than directed to the target tissue, the liver, and thus leads to a higher COHb level. Conversely, orally administered MC, which is absorbed through the GI tract, is metabolized in liver. Therefore, most of the released CO is centrally located within the liver rather than being bound to COHb. Thus the data indicates that CO generating compounds can be the choice vehicle to deliver potentially therapeutic CO into inflammatory areas in order to inhibit unregulated immune responses. CO generating compounds can be a family of immunosuppressive drug candidates which control allograft rejection and autoimmune diseases.

### Example 3

### CO-Mediated Protection Against Ischemia/Reperfusion Injury

Ischemia/reperfusion (I/R) insult is an antigen-independent component of the harvesting injury in orthotopic liver transplantation, and remains one of the major limitations of this procedure. Farmer et al., Transplantation Reviews 14(2): 106-116 (2000). The extent of liver damage due to I/R ranges from reversible changes with elevation of liver enzymes to severe injury resulting in cell death and ultimate liver failure. Previous studies have shown that upregulation of HO-1 can protect liver and heart cells from the oxidative stress caused by ischemic and reperfusion insult. Kato et al., Am. J. Transplant. 1:121-28 (2001); Katori et al., Transplantation (in press). To better understand the mechanism of HO-1 mediated protection against I/R injury, this study was designed to test the effects of HO byproduct CO on cold I/R injury in an ex-vivo isolated perfusion rat liver model.

Materials and Methods

Animals. Male Sprague Dawley (SD) rats weighing between 300-350 g (Harlan Sprague Dawley, Indianapolis, Ind.) were used. Animals were fed standard rodent chow and water and libitum and cared according to guidelines approved by the American Association of Laboratory Animal Care.

Isolated perfusion liver apparatus. An isolated perfusion liver apparatus was used, as described in Amersi et al., supra, and Maulik et al., Circulation 94:398-406 (1996). In brief, syngeneic rat blood, obtained for each experiment from four donor animals, was diluted to a hematocrit of 15% with Krebs Ringer Bicarbonate Buffer (mM: NaCl 118, KH₂POF 1, MgSO4 0.9, CaCl₂ 2.5, dextrose 11.1, and NaHCO₂ 25), and maintained at pH of 7.4. The perfusate was pumped from a heated reservoir that warmed the perfusate to 37° C. through silastic tubing oxygenator connected to a flow meter that measured portal vein blood flow (Cole Palmer Instruments, Chicago, Ill.). Portal pressure was kept constant via a pressure monometer connected to a T fitting in the portal vein canula. The outflow cannula in the inferior vena cava drained into an outflow reservoir. During the experiment, pH, temperature and oxygenation were kept constant.

Ex vivo cold ischemia model. SD rats underwent isoflourane anesthesia and systemic heparinization. After skeletonization of the liver, the portal vein, the inferior vena cava and the common bile duct were cannulated, and the liver was flushed with 10 ml of University of Wisconsin (UW) solution. The livers were then stored for 24 h at 4° C. in UW solution, followed by ex-vivo reperfusion for 1-2 h on an isolated perfusion liver apparatus. Portal vein blood flow, pressure, and bile production were recorded every 30 min. Blood samples were collected at 30 min intervals and serum glutamic oxaloacetic transaminase (sGOT) levels were measured using an autoanalyzer from ANTECH Diagnostics (Irvine, Calif.). At the conclusion of experiment, a portion of the liver was snap frozen for mRNA extraction/Western blot analyses; the remaining samples were fixed in formalin for H&E staining.

The role of CO—HO-1 pathway in hepatic I/R injury was studied in five major treatment groups (n=4.8 rats/group). In Group 1, the extent of I/R injury was contrasted between livers perfused ex-vivo with blood saturated with CO (300 parts per million [ppm]; 0.03% balanced air) vs. air alone (21% O₂). As NO may enhance HO-1 expression (Brouard et al., supra), we then investigated a link between the two gaseous molecules in Group 2 livers, which were perfused with CO supplemented with 25 mM NG-nitro-L-arginine methyl ester hydrochloride (L-NAME; Sigma Chemicals, St. Louis, Mo.), an inducible NO inhibitor. Because biological functions of CO have been linked to the generation of cGMP, an attempt was made in Group 3 to inhibit guanyl cyclase by perfusing livers with CO plus 10 mM 6-anilino-5,8-quinalinedone (LY-83583; Calbiochem, San Diego, Calif.), a cGMP analogue. To analyze as to whether exogenous CO can substitute for HO-1 in preventing I/R insult, Group 4 rats were treated 24 h prior to liver procurement with ZnPP)(1.5 mg/kg/i.p; Porphyrin Products, Logan Utah), an HO-1 inhibitor, followed by ex vivo perfusion with CO. It has been demonstrated that CO exerts anti-apoptotic effects that are dependent on the activation of p38 MAPK signal transduction pathway. Brouard et al., supra. Therefore, Group 5 rats were pre-treated 60 min before harvest with p38 MAPK inhibitor, SB203580, a pyridinyl imidazol (25 mg/kg orally; Sigma). In addition, prior to reperfursion with CO, SB203580 (20 μM) was added to the perfusate.

Histology. Liver specimens were fixed in a 10% buffered formalin solution and embedded in paraffin. Sections were made at 4 μm and stained with H&E. The histologic severity of I/R injury was graded using International Banff Criteria (Int'l Banff Schema Conference Worksheet, The Third Int'l Banff Conference on Allograft Pathology, June 21-25 (1995)). Using these criteria, lobular disarray and ballooning changes are graded from 1-4, where no change is given a score of 1 and severe disarray or ballooning changes are given a score of 4.

Myeloperoxidase (MPO) assay. MPO is a naturally occurring constituent of neutrophils and is used as a marker for neutrophil infiltration. Frozen tissue samples were thawed and suspended in an iced solution of 0.5% hexadecyltrimethyl-ammonium (Sigma) and 50 mMol potassium phosphate buffer solution (Sigma) with pH adjusted to 5. Samples are homogenized for 30 sec, centrifuged at 15,000 rpm for 15 min at 4° C. 0.1 ml of the supernatant was then mixed in solution of hydrogen peroxide-sodium acetate and tetramethyl benzidine (Sigma). The change in absorbance at 460 run was measured with a Beckman DU spectrophotometer (Beckman Institute, Fullerton, Calif.). The quantity of enzyme degrading 1 μMol peroxide per minute at 25° C. per gram of tissue was defined as one unit of MPO activity.

Western blots. Protein was extracted from liver samples with PBSTDS buffer (50 mM Tris, 150 mM NaCl, 0.1% SDS, 1% sodium deoxycholate, and 1% triton X-100, pH 7.2). Proteins (30 μg/sample) in SDS-loading buffer (50 mM Tris, pH 7.6, 10% glycerol, 1% SDS) were subjected to 12% SDS-polyacrylamide gel electrophoresis and transferred to nitrocellulose membrane (Bio-Rad, Hercules, Calif.). The gel was stained with coomassie blue to document equal protein loading. The membrane was blocked with 3% dry milk and 0.1% Tween 20 (USB, Cleveland, Ohio) in PBS and incubated with rabbit anti-rat HO-1 or iNOS polyclonal Abs (SangStat, Fremont, Calif.). Relative protein quantities were determined using a densitometer (Kodak Digital Science 1D Analysis Software, Rochester, N.Y.).

HO-1 Enzymatic Activity. Livers were homogenized on ice in a Tris-HCl lysis buffer (pH 7.4) containing 0.5% Triton X-100 and protease inhibitors. Samples were frozen in small aliquots until use. Homogenates (100 μl) were mixed with 0.8 mM NADPH, 0.8 mM glucose-6-phosphate 1.0 unit G-60P dehydrogenase, 1 mM MgCl₂ and 10 ml purified rat liver biliverdin reductase at 4° C. The reaction was initiated by the addition of hemin (final concentration 0.25 mM). The reaction mixture was incubated at 37° C. in the dark for 15 min. At the end of incubation period, any insoluble material was removed by centrifugation and supernatants were analyzed for bilirubin concentration. An extinction coefficient of 40 mM⁻¹ cm⁻¹ at A 460-530 was used to calculate the amount of bilirubin formed. Controls included naive samples in the absence of the NADPH generating system and all the ingredients of the reaction mixture in the absence of graft homogenates. Biliverdin reductase was purified from rat liver, as described in Browne and Ultrich, Mol. Pharmacol. 32:497-504 (1987).

ELISA for HO-1 protein expression. Livers were homogenized on ice in a Tris-HCl lysis buffer (pH 7.4) containing 0.5% Triton X-100 and protease inhibitors. Flat-bottom microtiter 96-well plates (Nunc) were coated with 7 μg/ml anti-HO-1 mAb (OSA-111, Stressgen, Canada) in PBS for 18 h at room temperature. Unbound Ab was removed by washing (wash buffer: 0.05% Tween 20 in 50 mM phosphate buffer, pH 7.5) and remaining binding sites were blocked by incubation with a 5% BSA/PBS solution (1 h). Recombinant HO-1 (SPP-730) and tissue homogenate were diluted in assay diluent (0.5% BSA/0.05% Tween 20/PBS) and incubated in anti-HO-1 mAb coated wells for 1 h at room temperature. Subsequently, plates were washed three times with wash buffer and incubated with rabbit anti-HO-1 polyclonal antibody (SPA-895, Stressgen; diluted 1:1000 in assay diluent) for 30 min at room temperature. Bound rabbit IgG was detected with a donkey anti-rabbit (gG-HRP conjugate (711-035-152, Jackson Research Laboratories; diluted 1:8000 in assay diluent). Unbound secondary Ab was removed by washing and bound HRP was detected using 1 mg/ml OPD in substrate buffer (0.1% H₂O₂, 0.1 M citric acid, 0.2 M Na₂HPO₄, pH 5.0). The color reaction was stopped with 1 M HCl and the optical density at 490 nm was measured.

Statistics. For statistical analysis, comparisons between the groups were done using repeated measure analysis of variance (ANOVA). If differences were established, we used the Tukey-Fisher Least Significance (LSD) criterion for judging statistical significance where p values of less than 0.05 were considered statistically significant. The values are expressed as mean±SEM.

Results

The effects of CO in an ex-vivo rat liver model of cold ischemia followed by reperfusion. In order to determine if the amelioration of I/R injury by HO-1 is mediated through the HO-1-CO downstream signaling pathway, portal vein resistance, bile production and sGOT levels were measured in rat livers that underwent 24 h of cold preservation followed by ex vivo 2 h perfusion with blood supplemented with CO (300 ppm balanced air) vs. air alone (21% O₂).

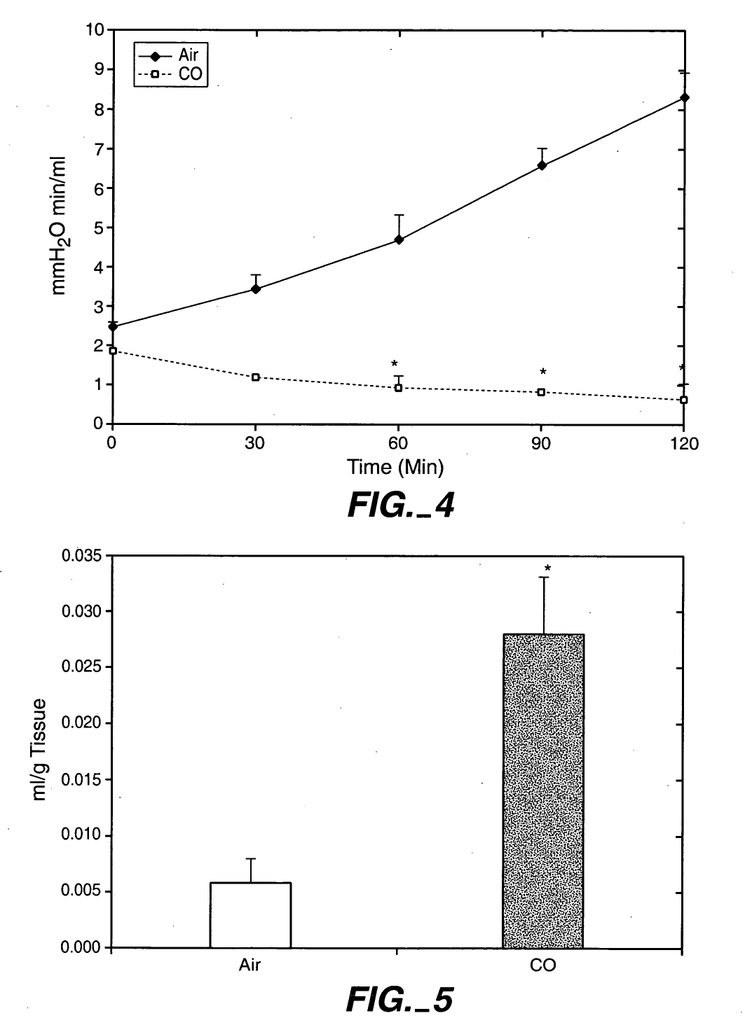

Portal vein resistance (pressure/flow) is affected by sinusoidal congestion and hepatocyte injury. Addition of CO to the perfusate significantly decreased (p<0.001) portal resistance (mmH₂O/min/ml) throughout the 2 h reperfusion period, as compared with controls (FIG. 4). Further, as shown in FIG. 5, CO-treated livers produced significantly more bile (ml/g tissue weight), as compared with livers perfused with blood exposed to air alone (p<0.005).

Next, we determined whether perfusion with CO ameliorated hepatocyte injury, as measured by sGOT release. Livers perfused with CO exhibited significantly lower (p<0.0001) sGOT levels (IU/L), as compared with control livers perfused with air alone (1 h: 79±14 vs. 362±51; 2 h: 163±27 vs. 497±31, respectively; data not shown).

The effects of CO on the severity of histologic features of I/R injury was evaluated by Banff's criteria. Control livers perfused with blood supplemented with air demonstrated extensive centrilobular ballooning and necrosis in association with sinusoidal and central vein congestion at 1 h (score=3.6±0.25) and 2 h (score=4.0 ±0.0) of reperfusion. In marked contrast, livers perfused with adjunctive CO for 1 h exhibited overall preservation of hepatic architecture without central vein or sinusoidal congestion, and an absence of centrilobular ballooning or necrosis (score=1.2±0.31). Livers perfused with CO for 2 h demonstrated patchy centrilobular ballooning and minimal necrosis with only mild vascular congestion and centrilobular pallor (score=2.0±0.12).

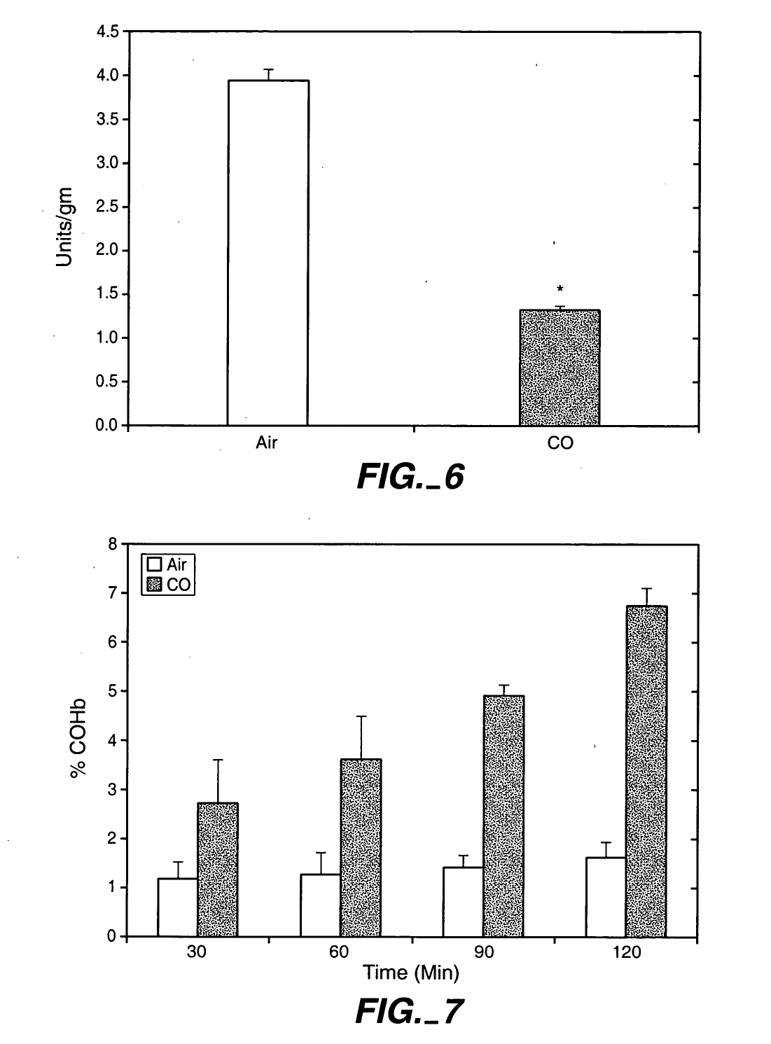

To study the mechanism of CO-mediated cytoprotective effects against I/R injury, the MPO assay was employed to determine neutrophil activity in liver tissue at the conclusion of 2 h of reperfusion. As shown in FIG. 6, control livers demonstrated a significant increase in MPO activity (4.0 U/mg), as compared with livers that were perfused with CO (1.3±0.2; p<0.04).

The protective effects of CO correlated with serial COHb measurements (FIG. 7). The concentration of COHb in blood exposed to CO for 1 h of 3.64±0.32% increased to 6.79±1.47% after 2 h of perfusion with CO. This value was significantly higher, as compared with livers perfused with air alone for either 1 h (1.27±0.19% COHb; p<0.01) or 2 h (1.59±0.13% COHb; p<0.002).

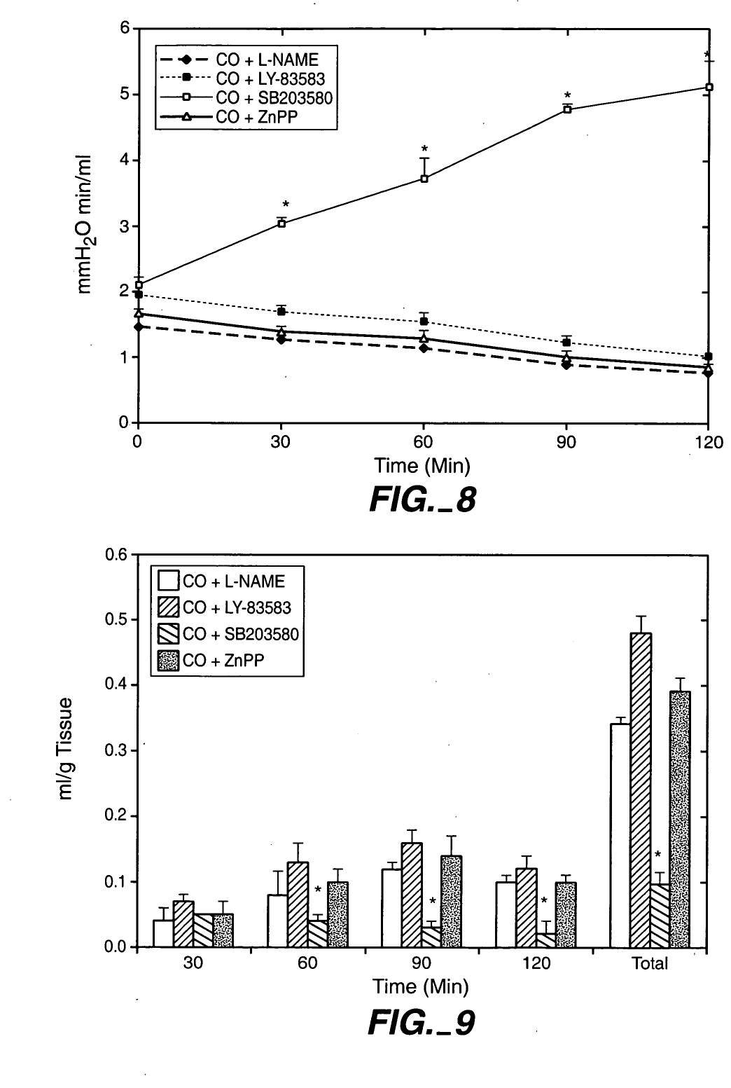

Effect of exogenous CO on hepatic I/R injury is through an NO-independent pathway. CO has been shown to directly bind to the heme moiety of the NO synthase enzyme, and to modulate NO production. Dulkanchainun et al., Ann. Surg. 227:832-840 (1998). Therefore, we investigated whether the amelioration of hepatic I/R injury seen with CO was mediated through NO. At the start of reperfusion, 25 mM of L-NAME, a selective inhibitor of iNOS, was added to the perfusate with CO. Livers treated with L-NAME in the presence of CO showed a decrease in portal vein resistance (mmH₂O/min/ml) and an increase in bile production (ml/g tissue weight) similar to the effects seen with livers exposed to CO alone after 2 h of reperfusion (FIG. 8 and FIG. 9, respectively). Furthermore, sGOT release (IU/L) was also decreased (191±16), as in the CO alone treated group (163±27).

Effect of exogenous CO on hepatic I/R injury is through a cGMP-independent pathway. To further investigate the possible mechanism by which CO exerts its protective effects, we examined the effects of inhibition of the cGMP, which is known to contribute to endothelium dependent vasodilation. Suematsu et al., J. Clin. Invest. 96:2431-37 (1994). LY-83583, a cGMP analog that interferes with the action of the nucleotide was added to the perfusate with CO at the time of reperfusion. After 2 h of reperfusion, inhibition of cGMP after adjunctive use of LY-83583 had no significant effects on hepatic function, as compared with livers perfused with CO alone. Although portal blood resistance was slightly increased in the groups perfused with LY-83683 (FIG. 8; NS), bile production (FIG. 9) as well as sGOT levels (186±21 IU/L) were comparable between both groups.

Exogenous CO can substitute for endogenous HO-1 in preventing hepatic I/R injury. To investigate whether depression of endogenous HO-1 activity affects the ability of exogenous CO to protect against I/R injury, we administered ZnPP (1.5 mg/kg/i.p.), a known HO-1 inhibitor, 24 h prior to the harvest. Livers were then kept for 24 h at 4° C., and perfused for 2 h ex vivo, as described above. Significantly, livers pretreated with ZnPP exhibited similar functional features as those in the group perfused with CO alone, i.e. decreased portal vein resistance (FIG. 8), increased bile production (FIG. 9), and improved hepatocyte function, as measured by sGOT levels (202±11 IU/L). Indeed, these cytoprotective effects correlated with depressed HO-1 enzyme activity (nmol of bilirubin/mg/protein/min; n=3-4/group) in the ZnPP pretreatment group (0.95±0.06), as compared with CO only (2.25±0.18; p<0.01) or air only (1.37±0.11; p<0.02) perfusion groups (data not shown). Similarly, ELISA-assisted detection of HO-1 protein expression in liver samples (ng of HO-1/mg lysate; n=3-4/group), revealed markedly diminished HO-1 content in the ZnPP pretreatment group (0.85±0.62) as compared with CO only (7.51±2.13; p<0.01) or air only (1.28±1.46; p<0.01) perfusion groups (data not shown).

Expression of HO-1 and iNOS. Western Blot analysis showed that CO-mediated cytoprotective effects against hepatic I/R injury correlated with upregulation of HO-1 expression. HO-1 protein was accentuated ca. 3-fold at 2 h after perfusion with CO, CO+L-NAME, and CO+LY-83583, when compared to the control group (air alone) and the group treated with ZnPP (data not shown). Analysis of iNOS expression using Western Blot resulted in no detectable bands in the CO, CO+L-NAME, CO+LY83583, and the CO+ZnPP treated groups; however a low density band was detected in the control livers after 2 h of reperfusion with air alone.

CO prevents hepatic I/R injury through the activation of p38 MAPK. As others have shown that CO prevents endothelial cell apoptosis via the activation of p38 MAPK transduction pathway (Brouard et al., supra), we investigated whether this mechanism played a role in our model. Livers treated with SB203580, a pyridinyl imidazol p38 MAPK inhibitor, in the presence of CO showed a significant increase in portal vein resistance (p<0.025) and produced significantly less bile (p<0.01), as compared with livers perfused with CO alone after 2 h of reperfusion (FIG. 8 and FIG. 9, respectively). In addition, sGOT release was increased (352±21 IU/L), when compared to the CO monotreatment group (163±27 IU/L; p<0.05). This data supports the contention that CO mediated protective effects against I/R injury are through activation of p38 MAPK signaling pathway.

Histology. The I/R induced hepatocyte injury was also graded at the conclusion of a 2 h perfusion period by using Banff's Criteria. Livers treated with CO+L-NAME revealed overall preservation of hepatic architecture without central vein or sinusoidal congestion, and minimal centrilobular ballooning with no necrosis (score=1.5±0.25). Livers treated with the cGMP analogue plus CO revealed preservation of hepatic architecture without central vein or sinusoidal congestion, and no centrilobular ballooning/necrosis (score=1.25±0.25). Livers pretreated with ZnPP followed by perfusion with CO showed minimal centrilobular ballooning, congestion and necrosis (score=1.5±0.0). Finally livers treated with CO+SB2035890 showed moderate ballooning change with sinusoidal and central venous congestion (score=3.25±0.25).

As indicated by the above data, rat livers perfused for 2 hours ex vivo with CO following 24 hours of cold storage showed significantly decreased portal venous resistance and increased bile production, as compared with control livers. This correlated with improved liver function (sGOT levels), decreased neutrophil infiltration, and diminished histologic features of hepatocyte injury (Banff's scores). The CO-mediated cytoprotective effects were nitric oxide or cGMP-independent, but p38 mitogen activated protein kinase (MAPK)-dependent. Moreover, CO could substitute for endogenous HO-1 in preventing hepatic I/R injury through the activation of p38 MAPK. Thus, CO administration has potential therapeutic application in preventing hepatic I/R injury and expanding the liver donor pool for transplant recipients.

### Example 4

### Methylene Chloride Administration Prevents Apoptosis and Extends Liver Allograft Survival

Apoptosis, or programmed cell death, is critical for the homeostasis of the immune system, and plays a central role in the destructive phase of acute allograft rejection by cytotoxic T lymphocytes (CTLs). CTLs can utilize a variety of mechanisms to lyse target cells, including the CD95/FAS system. Ju et al., Proc. Natl. Acad. Sci. USA 91:4185-89 (1994). This study investigated the effects of CO as a downstream mediator of HO-1 in preventing CD95/FAS-mediated apoptosis and prolonging allogeneic OLT survival.

Materials and Methods

Generation of recombinant adenovirus (Ad) encoding Fas ligand (Ad-CD95), heme oxygenase 1 (Ad-HO-1) and β-galactosidase reporter gene (Ad-β-gal). The Ad-HO-1 was generated, as described in Shibahara et al., Proc. Nat'l Acad. Sci. USA 93:10393-98 (1985). Briefly, the 1.0 k bp rat HO-1 cDNA flanked by Xhol-Hind III sites was cloned into plasmid pAC-CMVpLpA. The resulting pAC-HO-1 plasmid was co-transfected with plasmid pJM17 into 911 cells. Homologous recombination resulted in a replication-defective Ad-HO-1. Recombinant AD-HO-1 clones were screened by Southern blots. Ad-CD95 and Ad containing E. coli β-galactosidase gene (Ad-β-gal) have been described. Ke et al., Transplantation 69:1690-94 (2000). Isolation, propagation, and titration of recombinant Ads were carried out in a usual way. See Graham et al., Virology 52:456-67 (1973).

Cell lines. All cell lines were obtained from American Type Culture Collection (ATCC, Rockville, Md.). Hela cells were maintained in Dulbecco's minimum essential medium (DMEM; GIBCO, Grand Island, N.Y.)+10% fetal bovine serum (FBS), and YAC-1 cells in RPMI 1640 (GIBCO)+10% FBS medium.

In vitro cytotoxicity assay. Hela cells and YAC-1 cells plated at 1×10⁵ cells/well were cultured overnight in 100 μl of DMEM+10% FBS. After washing three times, Ad-CD95 (at multiplicity of infection [MOI]=5, 10, and 20) and Ad-HO-1 or Ad-β-gal (at MOI 10) were added and incubated for 1 hr with 100 μl of DMEM without serum. The medium was then removed and changed to 100 μl of DMEM with 2% FBS for incubation 36-48 hr. After removing medium and washing cells three times, 10 μl of MTT (5 mg/ml, Sigma Chemical, St. Louis, Mo.) was added to each well and incubated for 4 hr. After removal of medium, 100 μl of isopropyl alcohol with 0.01% HCl was added. An enzyme-linked immunosorbent assay reader was used at OD of 550. The percent of cytotoxicity was calculated as: 1-OD experimental/OD control×100%.

In vitro apoptosis assay. Hela and YAC-1 cells, plated in 96-wells at 1×10⁵ cells/well, were cultured overnight in 100 μl of DMEM+10% FBS. After washing three times, Ad-CD95 (at MOI of 5, 10, and 20) and Ad-HO-1 or Ad-β-gal (at MOI 10) were added and incubated for 1 hr with 100 μl of DMEM without serum. The medium was then removed and changed to 100 μl of DMEM with 2% FBS for 36-48 hr incubation. After removing medium and washing cells three times with PBS/1% BSA, 100 μl/well of a freshly 4% paraformaldehyde solution was added to cells and incubated for 1 hr. Then, 100 μl/well of permeabilisation solution (0.1% Triton X-100 in 0.1% sodium citrate) was added for 2 min on ice. After washing two times with PBS, cells were added with 50 μl/well TUNEL (terminal deoxynucleotidyl transferase-mediated dUTP nick-end labeling, see below) reaction mixture (Roche Molecular Biochemicals, Germany) and incubated for 1 hr at 37° C. in a humidified atmosphere in the dark. Cells were washed two times with PBS and then analyzed by fluorescence microscopy. The results were scored semi-quantitatively by averaging the number of apoptotic cells per microscopic field at 200× magnification. A minimum of six fields was evaluated per sample. Each experimental group was run in triplicate. All data are expressed as mean±SD.

Ad-HO-1 transduction in OLT model. Male Dark-Agouti (DA; RT1³) and Lewis (LEW; RT1′) rats of 10-16 weeks of age were purchased from Harlan Sprague Dawley, Inc. (San Diego, Calif.), and maintained under conditions approved by the UCLA Chancellor's Animal Research Committee (CARC). All animals were housed in microisolator cages in a virus free facility and fed laboratory chow ad libitum. Orthotopic liver transplants were performed between DA donors and LEW rat recipients, as described previously. Amersi et al., supra; Kato et al., Am. J. Transplant 1:121-28 (2001). Ex-vivo gene transfer into liver grafts was performed during cold preservation (4° C.) via perfusion of the portal vein with 2 ml of cold lactated Ringer's solution containing 5×10¹⁰ pfu (plaque-forming unit) of Ad-HO-1. Control grafts were perfused with 5×10¹⁰ pfu of Ad-β-gal. Animals were followed for survival. Separate groups of recipients were sacrificed at day 3, 7 and 10 post-transplant, OLTs were harvested for histological evaluation, whereas blood samples were collected for measurement of sGOT levels.

Methylene chloride treatment in OLT model To investigate whether CO represents a functional downstream HO-1 mediator in this system, methylene chloride was used as a carbon monoxide generator. As indicated above, the metabolism of MC is known to result in the exclusive production of CO₂ and CO. Gargas et al., supra. LEW rats transplanted with DA livers were fed with methylene chloride (500 mg/kg) 2 hr prior to the transplant, followed by a 2-week post-transplant course (500 mg/kg/day). The blood CoHb levels in experimental animals were measured at day 0, 5, and 10. Animal survival was screened and OLTs were analyzed histologically.

Histology. Liver allografts were harvested at day 3, 7 and 10 post-transplant. The tissue was sliced into small pieces, preserved in 10% neutral-buffered formalin, cut into 5-μm section, and stained with hematoxylin and eosin (H&E) by standard methods.

In vivo detection of apoptosis. A commercial in situ histochemical assay (Klenow-FragEL, Oncogene Research Products, Cambridge, Mass.) was performed to detect the DNA fragmentation characteristic of apoptosis in formalin-fixed paraffin-embedded tissue sections. In this assay, Kienow binds to exposed ends of DNA fragments generated in response to apoptotic signals and catalyzes the template-dependent addition of biotin-labeled and unlabeled deoxynucleotides. Biotinylated nucleotides are detected using a streptavidin-horse radish peroxidase (HRP) conjugate. Diaminobenzidine reacts with the labeled sample to generate an insoluble colored substrate at the site of DNA fragmentation. Counterstaining with methyl green aids in the morphological evaluation and characterization of normal and apoptotic cells. The results were scored semi-quantitatively by averaging the number of apoptotic cells per microscopic field at 200× magnification. Six fields were evaluated per tissue sample. All data are expressed as mean±SD.

Results

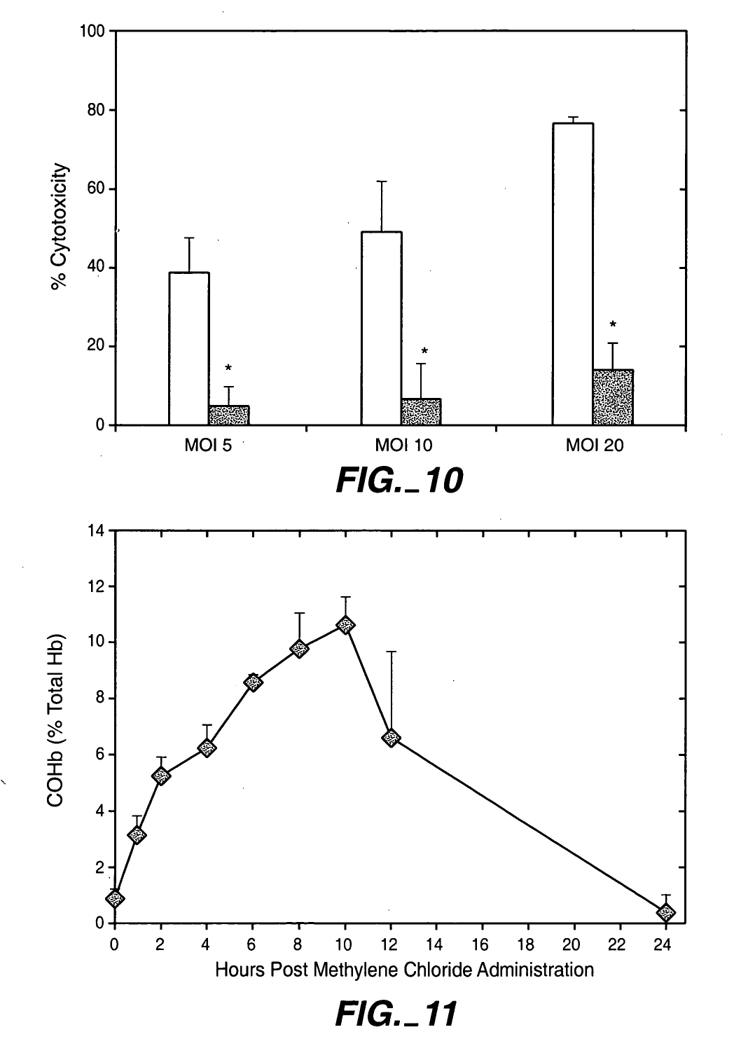

Ad-HO-1 gene transfer prevents CD95/Fas-mediated apoptosis in vitro. Cytotoxicity assay and TUNEL staining were used to analyze the effects of Ad-based HO-1 overexpression in vitro. As shown in FIG. 10, CD-95-mediated cytotoxicity to Fas-bearing YAC-1 target cells was consistently diminished in Ad-CD95+Ad-HO-1 transfected group, as compared with Ad-CD95+Ad-β-gal control. Indeed, at MOI of 5, 10 and 20, the cell death rate of 39%, 49%, and 76.5% in controls was significantly (p<0.001) higher as compared with 4.5%, 7% and 14% cell death in Ad-CD95+Ad-HO-1 group. In agreement with the results of the cytotoxicity assay, the number of TUNEL+apoptotic YAC-1 cells in Ad-CD95+Ad-β-gal group (211.5±76) was significantly (p<0.001) increased, as compared with Ad-CD95+AD-HO-1 group (36.5±14) (data not shown).

Ad-HO-1 gene transfer prolongs allogeneic OLT survival, ameliorates histological signs of acute rejection, and improves hepatic function. Untreated LEW rats died within 10 days following orthotopic transplantation of DA livers (see Table 1 below). Transfection of DA livers with Ad-β-gal did not affect animal survival after transplantation (mean survival time [MST]±SD=9.5±0.5 days; n=6). However, the survival of Ad-HO-1 transfected OLTs increased significantly to >32±42 days, with 2 out of 12 livers maintained for >120 days (Table 1). The X-gal positive staining after Ad-β-gal transfection was ca. 90%, 80% and 70% at day 3, 7 and 10 post-transplant, respectively (mean, 2-3 animals/group) OLTs in Ad-β-gal control group showed progressive signs of severe acute rejection, with necrosis, hemorrhage, and less than 25% of the hepatic parenchyma viable by day 10 post-transplant. In contrast, the corresponding OLT samples in the Ad-HO-1 group exhibited mild to moderate rejection, with dense inflammatory infiltrate, but more than 90% of parenchyma preserved. We then analyzed sGOT levels as a functional measure of OLT function. At day 10 post-transplant, sGOT levels (IU/L) were decreased in AD-HO-1 gene transfer group (412+105), as compared with Ad-1-Gal controls (1208+61 1; p<0.05). Ad-HO-1 gene transfer prevents apoptosis and upregulate the expression of anti-apoptotic molecules in allogeneic OLTs. By day 10, liver allografts in the Ad-1-gal group showed hepatocellular apoptosis with dense nuclear margination (64±25 of TUNEL+cells/field). In contrast, the number of apoptotic cells in allogeneic OLTs that underwent AD-HO-1 gene transfer remained within background levels (0.8±0.7 of TUNEL+cells/field; p<0.05).

Upregulation of endogenous CO prolongs allogeneic OLT survival. To investigate whether CO represents an important downstream HO-1 mediator in the rejection cascade, OLT allograft recipients were treated with methylene chloride (500 mg/kg/day×14 days). This regimen was well tolerated and no side effects were noted. The CoHb blood levels rose from 1.3±0.1% at the start of experiment (day 0), to 5.8±0.2%, and 6.8±0.5% at day+5 and +10, respectively (mean±SD; n=2-3 measurements/group). All untreated LEW rats died within 10 days after transplantation of allogeneic DA livers (Table 1 below). In contrast, OLT survival increased significantly after post-transplant feeding with methylene chloride (MST±SD=>47±46 days), with two out of seven rats surviving>120 days. By day 10, OLTs in untreated recipients showed severe acute rejection, with dense inflammatory infiltrate, portal/central veins showing necrotizing endothelitis, and less than 10% of the hepatic parenchyma viable. In contrast, OLTs harvested from methylene chloride-treated hosts showed a mild to moderate inflammatory infiltrate and central vein endothelitis, indicating mild to moderate rejection, and more than 80% of parenchyma well preserved. Methylene chloride-based CO delivery in vivo significantly reduced apoptosis in allogeneic OLTs at day 10 post-transplant from 61+25 of TUNEL+cells in untreated controls to 4.5±2.3 of TUNEL+cells in methylene chloride-treated rats (p<0.0025).

TABLE 1

| | | | Mean | |
|---|---|---|---|---|
| Treatment Protocol | Recipient Survival | N | (days) | P < |
| no treatment | 8, 9 (× 3), 10, 10 | 6 | 9.2 | |
| Ad-β-gal (livers perfused; | 9 (× 3), 10 (× 3) | 6 | 9.5 | |
| 5 × 10¹⁰ pfu/ml | | | | |
| Ad-HO-1 (livers perfused; | 12, 12, 13, 13, 14 (× 5), | 12 | >32 | |
| 5 × 10¹⁰ pfu/ml | 17, >120, >120 | | | |
| methylene chloride | 14, 17, 18, 21, 21, | 7 | >47 | |
| (recipients treated; | >120, >120 | | | |
| 500 mg/kg/d × 14 d) | | | | |

As indicated by the above data, AD-HO-1 gene therapy prevented CD95/Fas-mediated apoptosis in vitro, while enhanced in vivo HO-1 expression via Ad-HO-1 gene therapy significantly prolonged animal survival after allogeneic OLT, decreased histological severity of acute rejection and preserved hepatocyte architecture, and also improved OLT function as measured by sGOT levels. Correspondingly, daily feedings of OLT recipients with methylene chloride alone and with no other immunosuppression uniformly prevented ca. 10 day acute OLT rejection and significantly prolonged animal survival, with ca. 50% of rat recipients surviving>3 weeks. Elevated levels of CoHb following methylene chloride administration (from ca. 1.3% in untreated rats to ca. 6.8% after 10-day treatment) were consistently obtained and the regimen was well tolerated. Moreover, methylene chloride administration depressed the frequency of TUNEL+cells at the graft site, consistent with the notion that the anti-apoptotic effect in the HO-1-CO downstream signaling pathway is important in suppressing the allograft rejection cascade.

Thus, the above studies with methylene chloride indicate that Ad-HO-1 mediated anti-inflammatory effects in liver allograft recipients depend, at least in part, on the generation of CO. The above data are in agreement with others (Brouard et al. (2000), supra; Sato et al., supra and Fujita et al., Nat. Med. 7:598-604 (2001)) that CO alone can fully substitute for HO-1 mediated cytoprotection. In addition to its ability to suppress cell apoptosis, CO can also ameliorate graft rejection by depressing the fibrynolytic axis (Fujita et al.), inhibiting platelet aggregation (Brune and Ullrich, Mol. Pharmacol. 32:497-504 (1987) and/or promoting vasodilation (Motterlini et al., Cir. Res. 83:568-77 (1998).

### Example 5

### Methylene Chloride Administration Inhibits Chronic Reiection

Chronic rejection is characterized by allograft arteriosclerosis, a diffuse, progressive narrowing of the graft vessels due to intima hyperplasia. Libby and Roper, Immunity 14:387-97 (2001). Both I/R injury and immune responses against incompatible MHC antigens expressed by endothelial cells are viewed as the initiating causes of the disease. Endothelial cell destruction and/or activation as well as leukocyte infiltration of the intima and the adventitia lead to abnormal proliferation and migration of VSMCs from their normal position in the media to the intima subendothelial space, and to abnormal vasoconstriction. Id. Unlike acute rejection, there has been little progress in reducing the rate of chronic rejection in the last decades and there is an urgent need for new treatment strategies.

HO-1 has been shown to suppress inflammation in pathological situations relevant to chronic rejection such as ischaemia/reperfusion injury (Amersi et al., supra), atherosclerosis (Ishikawa et al., Cir. Res. 88:506-604 (2001)), neointima formation following arterial injury (Togane et al., Am. J. Physiol. Heart Circ. Physiol. 278:623-32 (2000)) as well as xenogeneic (Soares et al. and Sato et al., supra) and allogeneic graft rejection (Woo et al. and Hancock et al., supra), indicating that HO-1 may protect from chronic rejection by acting on immune and non-immune components of the disease. More recently, CO was shown to suppress the pro-inflammatory phenotype associated with monocyte macrophage activation (Otterbein et al., supra), to protect a variety of cell types from undergoing apoptosis (Petrache et al., Am. J. Physiol. Lung Cell. Mol. Physiol. 278:L312-319 (2000); Brouard et al., supra), to suppress xenograft rejection (Sato et al., supra) and to depress fibrinolysis (Fujita et al., supra). The present example demonstrates the effects of CO delivery in a well-characterized and widely used model of chronic aorta allograft rejection (Libby, supra) using the carbon monoxide generating compound methylene chloride.

Materials and Methods

Animals and aorta transplantation. Transplantations were performed using 250 g inbred male Lewis 1W rats (LEW. 1W, haplotype RT1^{u}) as donors and LEW.1A rats (haplotype RT1^{a}) as recipients (CERJ, Le Genest St. Isle, France). These animals are completely mismatched for the entire MHC region. Animal procedures followed European guidelines for animal experimentation. The descending thoracic aortas were harvested, perfused with saline and anastomosed to the recipient's abdominal aorta below the renal arteries and above the aortic bifurcation. Anastomosis was performed in a termino-lateral fashion and the recipient abdominal aorta was ligated between the two-graft anastomosis. Grafted aortas were harvested 30 days after transplantation. One aorta segment was fixed with 10% formaldehyde for morphometric evaluation and another segment was embedded in OCT compound (Tissue Tek, Miles Laboratories, Elkhart, Ind.) and frozen in liquid nitrogen for immunohistological analysis.

MC (Sigma, St. Louis, Mo.) was diluted in olive oil and administered orally on a daily basis (from day 0 to 30) at 500 mg/kg. This dose saturates the cytochrome P-450 oxidative system, and yields maximal COHb values of 10% COHb in venous or aortic blood. Gargas et al. and Andersen et al., supra; Wirkner et al., Toxicol. App. PharmacoL 143:83-88 (1997). Previously published data has demonstrated that 500 mg/kg MC administered per os is rapidly absorbed, reaching a mean concentration in blood of 60-70 mg/ml, is metabolized with a half life of about 3 h and generates around 10% HbCO with a half life of around 2 h. Id. Previously published data has also shown that 500 mg/kg administration of MC for 4 weeks did not induce major body weight, biochemical or histological changes in rats. Kirschman, Fd. Chem. Toxic. 24:943-49 (1986); Dhillon and Von Burg, Toxicology Update 1:329-35 (1995). MC may show liver and central nervous system toxicity at higher doses and/or longer exposures. Id. COHb levels were evaluated in heparinized venous blood using the VOXimeter sensor (A-VOX Systems, San Antonio, Tex.) and expressed as the percentage of total hemoglobin. Bicarbonates, soluble CO₂, total CO₂ and pH were measured using standard clinical biochemistry techniques (Laboratory of Biochemistry, University Hospital of Nantes).

Recombinant adenovirus and gene transfer into the aorta. An adenovirus coding for HO-1 (AdHO-1) was constructed using the pAdEasy and pAdTrack-CMV system (He et al., Proc. Nat'l Acad. Sci. 95:2509-14 (1998)) in 293 cells. AdHO-1 contains an expression cassette with the human CMV promoter and the human HO-1 cDNA fused to a Flag sequence in its 3′ end. The non-coding adenoviral vector Add1324 has been previously described, David et al., Hum. Gene Ther 9:1755-68 (1998), and recombinant adenoviruses were purified as described therein. Recombinant adenoviruses were titered using a Replication Center Assay (RCA). The protocol, originally described for the titration of adenovirus-associated vectors (Salvetti et al., Hum. Gene Ther. 9:6950706 (1998)), was modified to allow the quantification of infectious adenoviral particles (IP). Briefly, 293 cells were seeded at 8×10⁴ cells/well in 48-well plates. The next day, they were infected with serially-diluted vectors. Cells were trypsinized 36 hours later and filtered through a Zetaprobe membrane (Biorad). Filers were then soaked in 0.5 M NaOH, 1.5 M NaCl for 5 nm, neutralized in 1 M Tris-HCl pH 7.0, 2×SSC, and finally incubated with a fluorescein-labeled nucleic probe hybridizing to the DNA binding protein gene. Quantification of infectious adenoviral particles was determined by counting the number of spots (corresponding to individual viral replication events) on infected 293 cells. Importantly, quantification by RCA yield titers equivalent to infectious unit (determined by immunofluorescence using an anti-DBP antibody). Donor aortas were harvested, recombinant adenoviruses (10¹⁰ IP in 200 μl of DMEM supplemented with 1% FCS) were infused into the lumen and both extremities were ligated. Aortas were then incubated for 45 min at 37° C. 5% CO₂, flushed with DMEM to remove non-incorporated adenoviruses and transplanted into recipients.

Histology and morphometric analysis. After formaldehyde fixation, aorta segments were embedded in paraffin and 5 μm sections were stained with hematoxylin-eosin-saffron (HES). Microscopic images were collected using a color camera. Image analysis processing was carried out in a blinded fashion using the Scion Image software (National Institutes of Health). In each section, the area within the lumen, internal and external elastic lamina were circumscribed manually and measured. The thickness of the intima was calculated using the equation: intima/(intima+media)×100 and expressed as a percent of intima thickening.

Gene transfer in endothelial cells (ECs) and Western blot analysis. Primary aortic rat ECs were incubated (37° for 90 min) with Add1324 or AdHO-1 (50 IP per cell) in DMEM supplemented with 1% FCS, washed and cultured for 30 h in medium supplemented with 10% FCS. Cells were washed in PBS, trypsinized and lysed in a buffer containing 1% SDS, 240 μg/ml AEBSF (Sigma) and 0.71 TIU/ml aprotinin (Sigma) in 10 mM Tris pH 7.4. Twenty pg of protein were boiled and loaded onto 10% SDS-polyacrylamide gels followed by electrophoresis and blotting onto nitrocellulose membranes. Membranes were then blocked (overnight, 4° C.) with PBS containing 0.1% Tween20 and 5% nonfat dry milk, incubated (2 h, room temperature) with a rabbit anti-HO-1 that reacts with HO-1 of both human and rat origin (Stressgen, Victoria, BC, Canada), a mouse Mab anti-Flag (clone M2) (Sigma, St. Louis, Mo.) or a mouse Mab anti-β-tubulin (Calbiochem, San Diego, Calif.). They were then washed and incubated (2 h, room temperature) with a HRP-labeled anti-rabbit or anti-mouse IgG antibody (Jackson Immunoresearch, West Grove, Pa.) and detected with enhanced chemoluminiscence (Amersham, Arlington Heights, Ill.) using x-ray films.

Immunohistological analysis. Immunohistology was performed on cryostat sections as previously described in detail. Guillot et al., J. Immunol. 164:5258-68 (2000). Immunohistological analysis of infiltrating leukocytes was performed at day 30 after transplantation using the following mouse Mab: a mixture of two anti-leukocyte CD45 Mabs (OX1 AND OX30), anti-monocyte/macrophage CD68 (ED1), anti-αβ TCR (R.7.3), anti-CD4 (W3/25), anti-CD8 α chain (OX8), anti-monomorphic class II MHC antigens (OX6), anti-CD25 (OX39) (all from ECACC, Wiltshire, UK), anti-CD54 (ICAM-1) (Seikagaku America Inc., Rockville, Mass.), anti-CD86 (B7.2) (Pharmingen, Franklin Lakes, N.J.) and an irrelevant mouse Mab (3G8, anti-human CD16). VSMCs were detected using a mouse anti-α human smooth muscle actin Mab (Sigma, St. Louis, Mo.). Slides were then incubated with a biotin-conjugated anti-mouse immunoglobulin antibody (Vector Laboratories, Burlingame, Calif.), followed by HRP-conjugated streptavidin (Vector Laboratories) and VIP substrate. IFNγ expression was analyzed using a hamster Mab (Genzyme, Cambridge, Mass.). The IP10 chemiokine was detected using a goat anti-IP10 antibody (Santa Cruz, Santa Cruz, Calif.). Rabbit antibodies were used to detect iNOS (Transduction Laboratories, Lexington, Ky.), HO-1 (Stressgen) and TGFβ1 (Promega, Madison, Wis.). Biotin-conjugated anti-hamster, anti-goat and anti-rabbit antibodies were from Jackson Immunoresearch. Binding of these antibodies was detected by incubation with HRP-conjugated streptavidin and VIP substrate. Tissue sections were counterstained with hematoxylin and lithium carbonate.

Expression of HO-1 after adenovirus-mediated gene transfer was confirmed on cryostat sections (20 μm) of aortas transduced with Add 1324 or AdHO-1 exactly as described before transplantation and cultured for 2 days in DMEM containing 10% FCS, following a previously-described technique allowing to keep the endothelium in a resting and viable condition after adenovirus-mediated gene transfer. Merrick et al., Transplantation 62(8):1085-1089 (1996); Merrick et al., Transplant. Immunol. 5:3-9 (1997). Aorta cryostat sections (20 μm) were fixed with 2% paraformaldehyde (20 min, room temperature), permeabilized with 0.1% triton and incubated (18 h, 4° C.) with 200 μl of biotin-conjugated anti-Flag or rabbit anti-HO-1 antibodies (10 μg/ml diluted in PBS with BSA 1%, rat serum 1% and triton X 100). Binding of these antibodies was detected as described above. All immunohistology experiments included as negative controls the 3G8 irrelevant Mab or control sera from the species used to detect inflammatory mediators.

Detection of alloantibodies. Donor LEW.1W splenocyts were incubated with heat-inactivated serum from ELW.1A recipients, serially diluted in PBS. Cells were then washed and simultaneously incubated with FITC-coupled donkey anti-rat IgG (Jackson Laboratories) and with a biotin-labeled anti-B cell Mab (clone OX33, ECACC). After washing, cells were incubated with phycoerythrin-coupled streptavidin. Serum levels of alloantibodies were determined by cytofluorimetry (FACScalibur, Becton Dickinson, San Jose, Calif.) and reported as the mean channel fluorescence (MCF) at each dilution of serum. A predominance of anti-donor MHC class II alloantibodies, as previously described in certain tolerance models (Cuturi et al., Eur. J. Immunol. 24:1627-31 (1994)), is detected by the binding of alloantibodies only to OX33 positive cells (B cells). The presence of anti-MHC class I alloantibodies results in labeling of OX33 negative (T cells) and positive cells. MCF of alloantibody binding to OX33 negative cells indicated the level of anti-MHC I alloantibodies.

Statistics. Statistical significance (P<0.05) was evaluated using ANOVA.

Results

Expression of HO-1 after adenovirus-mediated gene transfer and CO release after MC administration. The expression of HO-1 following infection with AdHO-1 was confirmed in cultured rat ECs and in aortas. Untreated rat ECs and Add1324-transduced cells showed low levels of endogeneous HO-1 expression whereas Ad-HO-1 transduced ECs displayed strong expression of HO-1 as detected by Western blot with anti-HO-1 and anti-Flag antibodies. Due to the presence of the Flag peptide, HO-1 expressed following AdHO-1-transfection has a higher molecular weight than endogenous HO-1 (33 vs. 32 kDa). The anti-Flag antibody displayed a band of the expected molecular weight only in AdHO-1 transduced EC despite a non-specific cross-reactivity in control cells. The enzymatic activity of HO-1 (the generation of bilirubin) was augmented in cells transduced with AdHO-1 compared to Add1324 transduced cells.

Expression of HO-1 was confirmed by immunohistology in aortas transduced with AdHO-1 using anti-HO-1 and anti-Flag antibodies. Expression of the HO-1-Flag molecule was absent in control adenovirus-treated tissue and anti-Flag antibodies. HO-1 expression by the endothelium was also detected in AdHO-1 but not Add 1324-transduced aortas by immunohistology on whole aorta fragments, using a previously described technique (Merrick et al., supra). HO-1 expression was detected up to day 10 in transplanted aortas and was absent at day 15. These results indicate that HO-1 vectorized by AdHO-1 was expressed following gene transfer into ECs and aorta.

Production of CO following metabolism of orally administered MC was confirmed by analysis of COHb blood levels at various time points (FIG. 11). Following administration of MC, COHb levels (mean of total Hb±SEM, n=4) rose from 0.8±0.3 to 10.6±1 within 10 hours and declined to normal levels within 24 hours after MC administration. As compared to values prior to administration, animals that received MC (500 mg/kg, n=5) and were analyzed at 4, 8, 10, 12, 16, 20 and 24 h did not show significant changes in blood bicarbonates, soluble CO₂, total CO₂ and pH values (data not shown). Therefore, CO₂ generated from MC was efficiently buffered by the carbonate system and then eliminated by respiration without physiological modifications. Consistent with previously published data (Kirschman and Dhillon et al., supra), MC-treated rats showed normal behavior, food consumption and no particular gross necropsy alterations. These results indicate that the effects of MC administration are the result of CO delivery and not of CO₂.

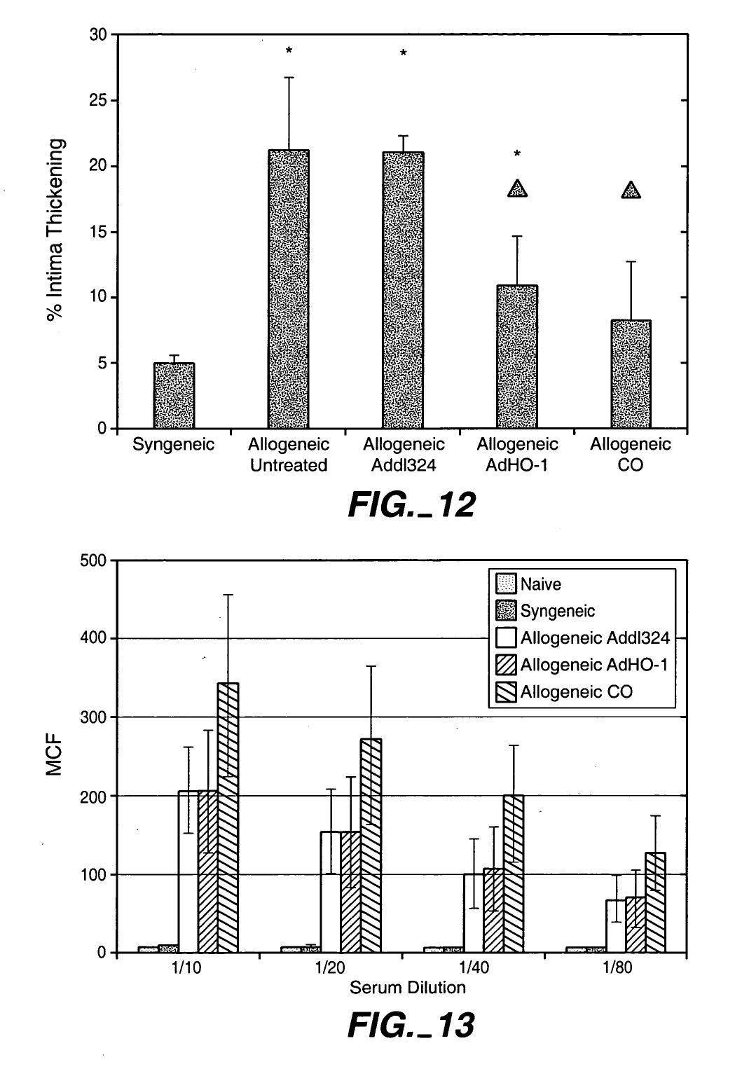

Expression of HO-1 after gene transfer and CO delivery reduces intimal thickening. Intimal thickening was of a similar magnitude and aspect in chronically rejected untreated and control-adenovirus-treated aortas (mean±SEM; 21.2±5.6%, n=4 and 21.1±1.2, n=5%, respectively) (FIG. 12). Intimal thickness in syngeneic grafts (4.8±0.7%, n=4) and non-grafted aortas was similar (data not shown). Gene transfer using AdHO-1 resulted in a significant reduction in intimal thickness was also observed in MC-treated recipients (8.3±4.5%, n=5).

Microscopic examination of HES stained untreated or control adenovirus-treated aortas revealed that intimal thickening was the result of cellular infiltration and extracellular matrix deposition. The media of chronically rejected aortas showed a reduction in cell density while the adventitia was heavily infiltrated with extracellular matrix deposition in their intimas. The adventitia of AdHO-1 treated aortas showed a clear reduction in infiltrating cells whereas those from MC-treated aortas displayed a moderate reduction.

These results indicate that gene transfer of HO-1 or administration of CO, one of the products of heme degradation by HO-1, decreases the development of chronic rejection lesions.

Gene transfer of HO-1 and CO delivery reduces cellular infiltration of the intima. Immunohistological analysis of syngeneic grafts revealed no leukocyte infiltration of the intima or adventitia. VSMCs were restricted to the media without any reduction in VSMCs density (Table 2). In allogeneic grafts treated with non-coding adenovirus, a large number of infiltrating leukocytes were observed in the intima and adventitia (Table 2). The majority of infiltrating CD45+leukocytes were CD68+macrophages and to a lesser extent T lymphocytes (Table 2). The T cell population contained more CD4+cells than CD8+cells (Table 2). As opposed to syngeneic aortas in which VSMCs were homogeneously distributed in the media, non-codant adenovirus-treated allogeneic aortas showed VSMCs in the intima and a reduced number in the media (Table 2).

TABLE 2

| | | CD45 | CD68 | TCRαβ | | | |
|---|---|---|---|---|---|---|---|
| Graft Type | Tissue | (leukocytes) | (macrophages) | (T cells | CD4 | CD8 | VSMCs |
| syngeneic | intima | − | ND | ND | ND | ND | − |
| | media | − | ND | ND | ND | ND | +++ |
| | adventitia | − | ND | ND | ND | ND | − |
| allogeneic | intima | +++ | +++ | + | ++ | + | ++ |
| Addl324 | media | + | − | − | − | − | ++ |
| | adventitia | +++ | +++ | ++ | ++ | + | − |
| allogeneic | intima | + | + | − | + | + | + |
| Ad-HO-1 | media | − | − | − | − | − | ++ |
| | adventitia | + | + | + | + | + | − |
| allogeneic | intima | ++ | ++ | − | + | + | − |
| CO | media | − | − | − | − | − | +++ |
| | adventitia | +++ | +++ | + | ++ | + | − |

Frequency of stained cells was graded as:
−, not present;
+, low;
++, moderate;
+++ frequent

As indicated in the above Table, aortas treated with AdHO-1-showed a marked reduction in intima and adventitia infiltration by total CD45+leukocytes, macrophages, T and CD4+cells compared to control adenovirus-treated aortas. AdHO-1 treated aortas showed a reduction in VSMCs in the intima but some areas of the media displayed reduced VSMCs density.

Similar to the effects observed in AdHO-1-transduced aortas, delivery of CO through treatment with MC reduced intima infiltration by total leukocytes, macrophages, T, and CD4+cells. However, this effect appeared to be less pronounced than in AdHO-1-treated aortas. In addition, CO treatment did not affect leukocyte infiltration of the adventitia (Table 2). On the other hand, the effect of CO on VSMCs was more pronounced than that observed for AdHO-1-treated aortas since VSMC distribution in aortas from MC-treated recipients was identical to VSMC distribution in syngeneic transplanted aortas: VSMCs were not detectable in the intima and the media showed a normal VSMC density (Table 2).

Gene transfer of HO-1 and CO delivery reduces the expression of activation markers and cytokines. Syngeneic aortas showed weak labeling of ICAM-1 on the endothelium, no labeling for B7.2 and MHC class II antigens and low to moderate labeling in the media and adventitia for IP10, TGFβ1 and iNOS (Table 3). Control-adenovirus-treated allogeneic aortas showed large numbers of cells in the intima and adventitia strongly expressing ICAM-1, B7.2 and MHC class II antigens (Table 3). IP10, TGFβ1 and iNOS expression was also increased in the intima and adventitia and additionally in the media of control adenovirus-treated aortas, as demonstrated in Table 3 below.

TABLE 3

| Graft Type | Tissue | CD54 (ICAM-1) | CD86 (B7.2) | MHC-II | IP10 | TGFβ | INOS |
|---|---|---|---|---|---|---|---|
| syngeneic | intima | 54 | ND | ND | ND | ND | − |
| | media | − | ND | ND | ND | ND | +++ |
| | adventitia | − | ND | ND | ND | ND | − |
| allogeneic | intima | +++ | ++ | +++ | + | ++ | +++ |
| Addl324 | | | | | | | |
| | media | − | − | − | ++ | + | ++ |
| | adventitia | +++ | + | +++ | ++ | ++ | +++ |
| allogeneic | intima | + | + | + | − | + | ++ |
| Ad-HO-1 | | | | | | | |
| | media | + | − | − | + | + | ++ |
| | adventitia | ++ | + | + | + | + | + |
| allogeneic | intima | ++ | + | ++ | + | + | ++ |
| CO | | | | | | | |
| | media | − | − | − | + | + | ++ |
| | adventitia | +++ | + | ++ | + | ++ | +++ |

Frequency of stained cells was graded as:
−, not present;
+, low;
++, moderate;
+++ frequent

AdHO-1-treated aortas displayed a reduced number of cells expressing ICAM-1, B7.2, MHC class II antigens. IP10, TGFβ1 and iNOS were expressed with less intensity compared to control adenovirus-treated aortas (Table 3). CO-treatment through MC administration moderately reduced the expression of ICAM-1, B7.2 and MHC class II molecules (Table 3). IP10 expression was reduced in the media and adventitia but not in the intima whereas TGFβ1 and iNOS expression was reduced in the intima but not in the media or adventitia (Table 3). IL-2 receptor (CD25) and IFNγ were detected in rare and dispersed cells of allografts without differences between the experimental groups and were not detected in syngeneic grafts (data not shown).

In conclusion, analysis of leukocytes and inflammatory mediators showed that AdHO-1-treated aortas displayed decreased intimal and adventitia inflammation whereas CO-treated aortas presented a less pronounced reduction of these inflammatory markers in the intima and no reduction in the adventitia. In contrast, the effect on VSMCs reduction in the intima and increase in the media, was more pronounced in CO than in Ad-HO-1-treated aortas.

The Ad-HO-1 effect could be explained by the production of biliverdin and bilirubin within Ad-HO-1 transduced EC, as well as iron depletion, thus inhibiting EC activation and therefore leukocyte adhesion and tissue infiltration. Simultaneously, CO diffusing from Ad-HO-1 transduced EC could act not only on adjacent EC and macrophages but also on VSMCs, inhibiting their apoptosis, proliferation and activation. The transient expression of HO-1 mediated by Ad-HO-1, which is undetectable at day 15 after transplantation, may explain a more efficient effect on the early leukocyte infiltration phase and a less pronounced effect on later VSMC proliferation. In contrast, methylene chloride therapy was administered continuously throughout the experiment and could have inhibited VSMC proliferation more effectively than leukocyte infiltration. Additionally, CO delivery may also produce higher levels of CO in the arterial wall compared to Ad-HO-1 gene transfer.

Analysis of alloantibody levels in recipients with grafts treated with AdHO-1-and after CO delivery. Alloantibodies are produced in secondary lymphoid organs and reflect CD4-dependent alloreactivity. Alloantibodies have been implicated in the development of chronic rejection in certain but not all models (Libby and Pober, supra). A predominance of anti-donor MHC class II alloantibodies has been previously described associated with long-term allograft survival (Cuturi et al., Eur. J. Immunol. 24:1627-31 (1994)). Recipients of aortas treated with Ad-HO-1 or receiving MC showed a profile of alloantibody binding to both T and B donor cells identical to recipients of control adenovirus-treated aortas, indicating no preferential production of anti-MHC class II alloantibodies (data not shown). Levels of anti-MHC class I alloantibodies showed no statistical differences between recipients grafted with control or AdHO-1 treated aortas or those exposed to CO after MC administration. (FIG. 13)

The fact that alloantibody levels were not decreased in recipients of Ad-HO-1-treated aortas or aortas treated with CO indicates that either alloantibodies do not play an important role in this model of chronic rejection or that HO-1 and CO inhibit downstream effects of alloantibodies. These results, together with the decrease in infiltration by leukocytes and production of pro-inflammatory mediators, suggest that HO-1 gene transfer or CO therapy mainly act through local immunosuppressive effects on effector mechanisms.

As demonstrated by the above data, both adenovirus-mediated HO-1 gene transfer into the endothelium of the aorta and CO delivery resulted in a significant reduction in intima thickness compared to control non-coding adenovirus-treated aortas. Aortas transduced with Ad-HO-1 or treated with CO showed a reduction in the number of macrophages, T cells and CD4+cells as well as in the expression of adhesion molecules, costimulatory molecules and cytokines, with the gene transfer displaying a more pronounced effect than the CO treatment. Conversely, CO inhibited VSMC accumulation in the intima and preserved the vascular media more efficiently than Ad-HO-1 treatment. Based on the observation that CO therapy using methylene chloride revealed an inhibition of chronic rejection similar to that obtained with Ad-HO-1, the above results suggest that CO can mediate protective effects associated with increased expression of HO-1.

### Example 6

### Therapeutic Effects of Methylene Chloride in a Rat Collagen-Arthritis Model

Collagen-induced arthritis (CIA) is a T cell-dependent animal model of rheumatoid arthritis. Trentham et al. J. Exp. Med. 146:857-68 (1977); Brahn et al., Arthritis and Rheumatism 37 (6):839-45 (1994). Within two weeks after immunization with type II collagen in Freund's incomplete adjuvant susceptible rats develop polyarthritis with histologic changes of pannus formation and bone/cartilage erosion. In addition, humoral and cellular responses to collagen type II occur in CIA as well as rheumatoid arthritis. Consequently, CIA is a useful and accepted animal model for rheumatoid arthritis that serves as an in vivo system for the exploration of inflammatory synovitis etiologies and for the investigation of potentially new therapeutic interventions.

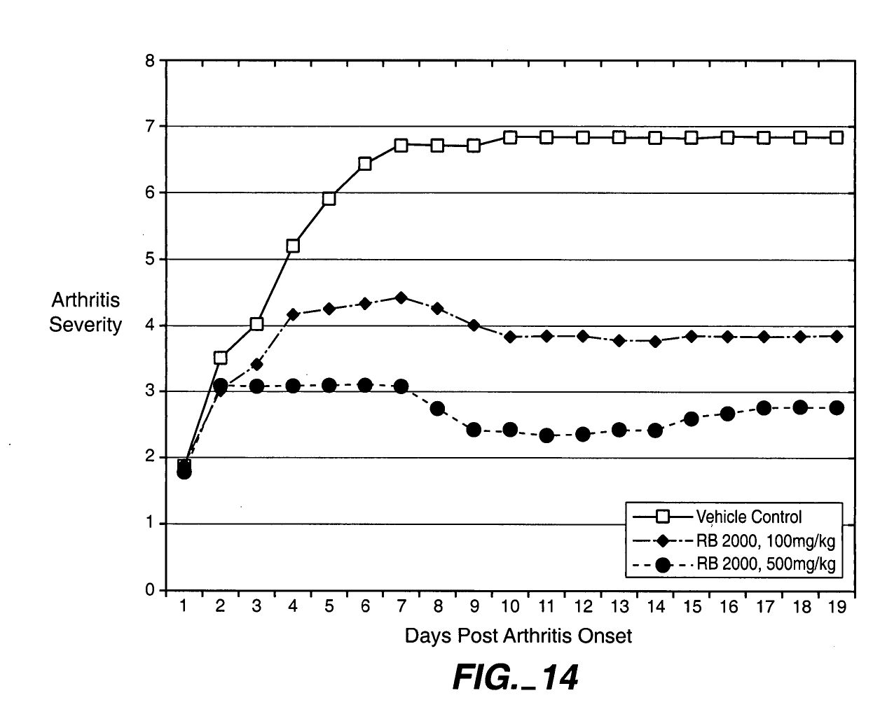

To assess the therapeutic potential of methylene chloride as a carbon-monoxide generating compound in this disease model, female Louvain rats weighing between 120 and 150 g were injected intradermally with 0.5 mg native chicken collagen type II solubilized in 0.1M acetic acid and emulsified in incomplete Freund's adjuvant. At the onset of disease (around day 10) animals were divided into three groups. One group was treated daily with vehicle, the second and third groups with 100 mg/kg/day and 500 mg/kg/day methylene chloride (p.o.). Severity of disease was evaluated daily using a quantification method based on standardized levels of swelling and periarticular erythema. Animals were sacrificed on day 28. As illustrated in FIG. 14, at the end of the study the arthritic score in vehicle treated animals was 6.8+/−0.7 (mean+/−standard error), 3.8+/−1.0 in animals treated with 100 mg/kg/day and 2.75 in animals treated with 500 mg/kg/day. Compared to control animals these differences were statistically significant (p<0.02).

A blinded analysis of bone erosion by X-ray confirmed the therapeutic effect of methylene chloride therapy. The X-ray score for limbs from vehicle treated animals was 4.8+/−0.7. Methylene chloride therapy with 100 mg/kg/day resulted in a score of 2.7+/−0.8, therapy with 500 mg/kg/day in a score of 1.8+/−0.7. This difference was statistically significant (p<0.05).

### Example 7

### Effect of Methylene Chloride Therapy on Neointimal Growth Following Carotid Wire Injury in the Atherogenic ApoE−/−Mouse

The accumulation of VSMCs in neointimal resulting from the migration and proliferation of medial VSMCs in response to endothelial damage is believed to be one of the main events involved in the initiation of atherosclerosis. Previously, carbon monoxide generated through heme oxygenase was shown to inhibit mitogen-induced proliferation of vascular smooth muscle cells (Togane et al., supra, Duckers et al., Nat. Med. 7(6):693-98(2001)).

The effects of CO generated though metabolic degradation of methylene chloride are investigated in an atherosclerotic mouse carotid intimal denudation model. Female C57BL/6 ApoE (−/−) mice (10-12 weeks old, n=12/group) are fed Western diet for 1 week prior to injury and 4 weeks after injury. On day 0 mice are anesthesized with ip injection of ketamine (80 mg/kg) & xylazine (5 mg/kg). The left carotid artery is isolated and two ligatures (6-O silk) are placed around the external carotid artery, ligatures are also placed around the common and internal carotid arteries. After the distal external carotid ligatures are tied, the carotid is incised with Vannas scissors proximal to the ligature. A curved flexible wire (0.35 mm/0.014 in diameter) is introduced into the external carotid and passed three times along the wall of the common carotid while being rotated. Upon removal of the wire the proximal carotid ligature is tied and the skin is reopposed with 6-O silk.

Methylene chloride (25, 100, 400 mg/kg/day) is administered intraperitoneally or orally starting on day-1 until day 28. A control group is treated with vehicle. On day 28 animals are sacrificed and after incision, the right and left common, external, and internal carotids are ligated. After sternotomy, common carotids are dissected further to the aortic arch. A 27-gauge needle is placed in the left ventricle and a systemic perfusion with phosphate buffered paraformaldehyde (100 mM, 4% wt/vol, pH 7.3) is performed at 100 mmHg via the left ventricular cannula. Subsequently, the common, internal and external carotid arteries are transected and removed. Specimens are dehydrated in ethanol and xylene, and embedded in paraffin. Tissue sections are stained and VSMC proliferation is assessed microscopically by histomorphometry. Methylene chloride therapy at 400, 100, and 25 mg/kg/day inhibits neointimal formation by 90%, 75% and 30% respectively.

### Example 8

### Haloforms as Carbon Monoxide Generating Compounds

The ability of the trihalomethanes iodoform and bromoform to act as carbon monoxide generating compounds was assayed in comparison with methylene chloride. As shown in FIG. 19, administration of 100 mg/kg of bromoform and iodoform resulted in a higher COHb level in comparison with the same amount of methylene chloride.

### Example 9

### Efficacy of Haloforms and Dihalomethanes in Arthritis Model

Iodoform and bromoform were compared with methylene chloride as alternative carbon monoxide generating compounds, using the rat collagen arthritis model described in Example 6 above. Briefly, arthritis was induced in a Lovain strain of rats by immunizing the animals with Collagen Type II in Incomplete Freund's Adjuvant. Digitized radiographs were obtained at the completion of the study on day 28. Pharmacokinetics were also determined and the synovial tissue from the inflamed joints of the treated animals was harvested on Day 16 or Day 28 post-immunization. Disease systems were generally evident by Day 10.

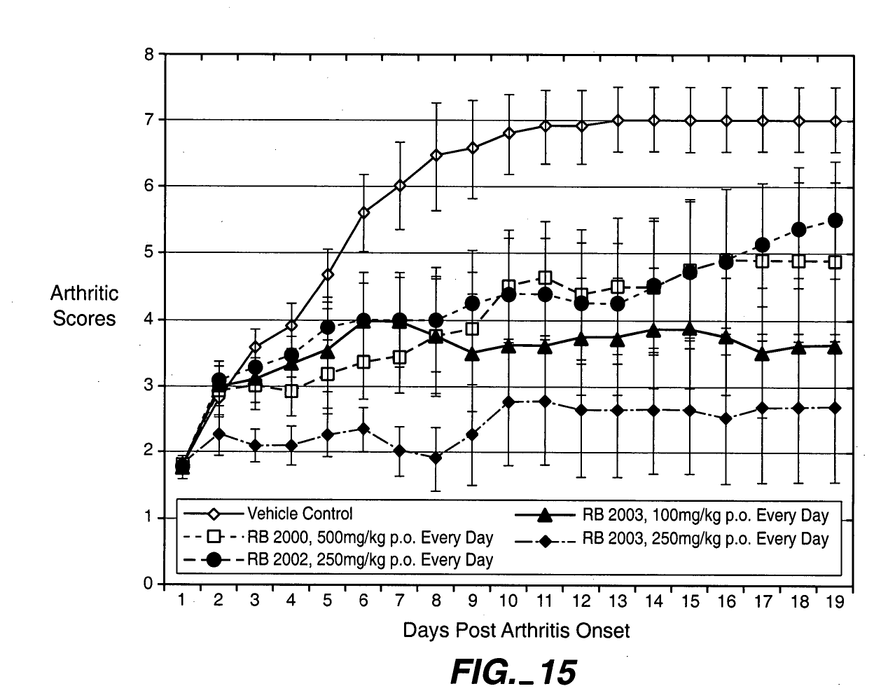

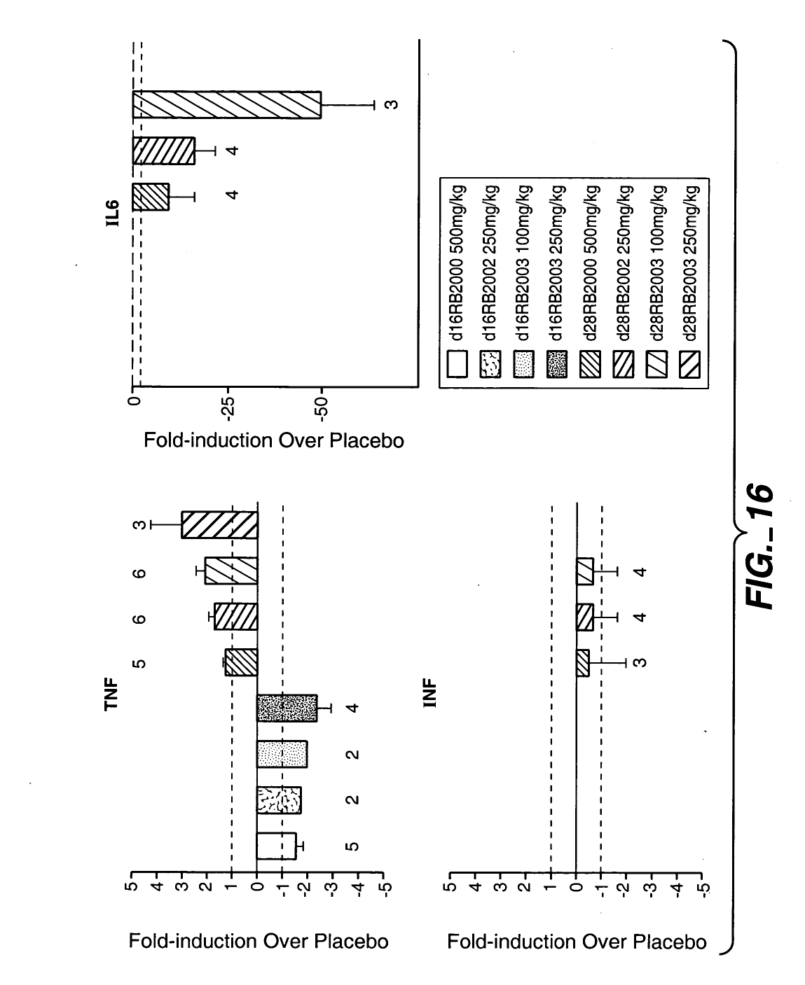

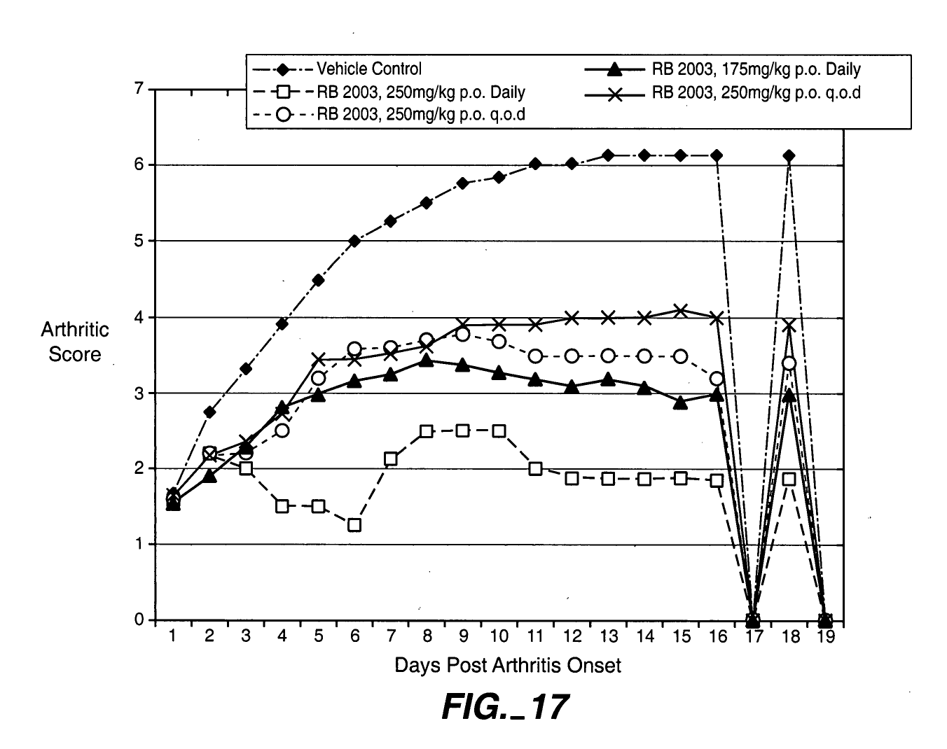

As shown in FIG. 15, both iodoform (RB2003) and bromoform (RB2002) exhibited superior efficacy in this model in comparison with methylene chloride (RB2000), and in the case of iodoform in particular the improved efficacy could be obtained using dramatically reduced amounts of generator compound. Both bromoform and iodoform significantly inhibited structural damage as determined by blinded radiographic scores, with iodoform acting in a dose-dependent fashion and appearing to be superior to bromoform, as shown in Table 4 below and in FIGS. 15 and 17:

TABLE 4
Day 28 Outcomes

| | Dose | Arthritis | | | |
|---|---|---|---|---|---|
| Agent | mg/kg | Score | p Value | X-Ray Score | p Value |
| Vehicle | | 6.08 ± 0.57 | | 3.58 ± 0.64 | |
| RB-2002 | 250 | 5.5 ± 0.86 | p = 0.07 | 2.75 ± 0.81 | p < 0.05 |
| RB-2003 | 100 | 3.62 ± 0.99 | p < 0.01 | 1.50 ± 0.80 | p < 0.001 |
| RB-2003 | 175 | 3.16 ± 0.99 | p < 0.005 | 1.90 ± 0.64 | p < 0.05 |
| RB-2003 | 250 | 1.85 ± 0.91 | p < 0.001 | 0.85 ± 0.45 | p < 0.005 |

Carboxyhemoglobin levels peaked within a few hours of administration but normalized within 24 hours without cumulative pharmacokinetic consequences. Messenger RNA (mRNA) from the treated animals was purified and specific mRNA levels for cytokines TNF, IL-6, and IFN-γ were quantitated by Quantitative RT-PCR. As shown in FIG. 16, on Day 28 mRNA levels for TNF were shown to be higher in both bromoform and iodoform treated rats as compared to rats treated with methylene chloride. The arthritic scores for the bromoform and iodoform treated rats were, however, significantly lower in comparison to the arthritic scores of the methylene chloride treated rats. The mRNA levels for IL-6 and IFN-γ were substantially lower in both the bromoform and iodoform treated rats as compared to rats treated with methylene chloride. These results suggest that inhibition of IL-6 and IFN-γ may be sufficient to decrease disease activity in the haloform treated rats.

### Example 10

### Efficacy of Haloforms and Dihalomethanes in Multiple Sclerosis Model

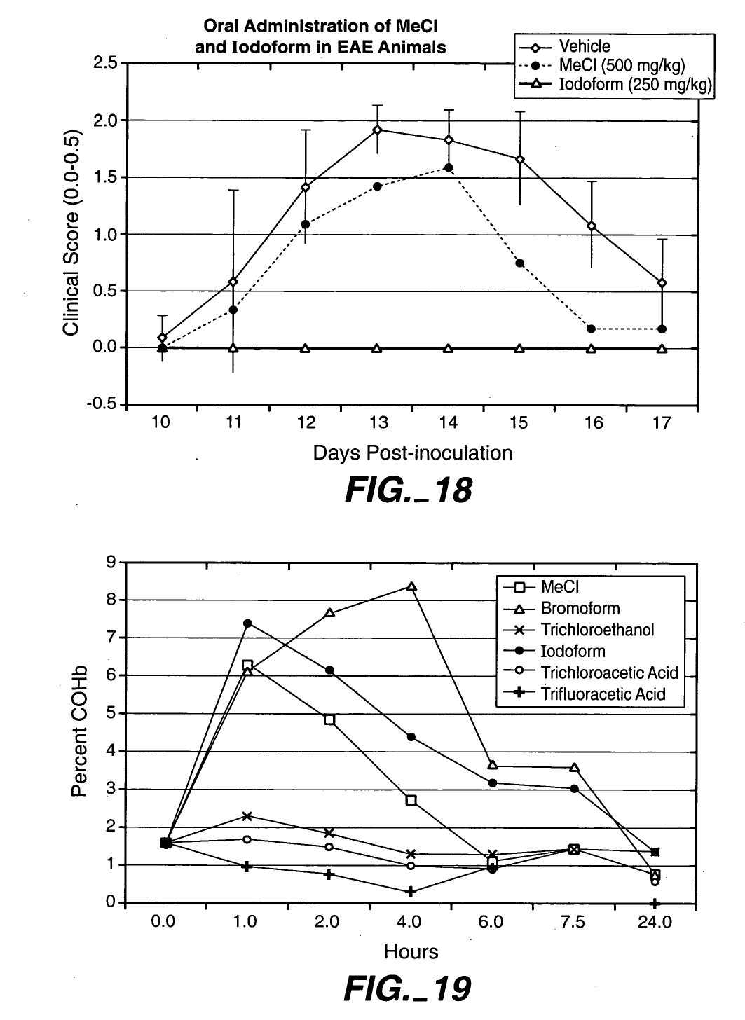

Multiple sclerosis is characterized by an autoimmune and inflammatory response directed against myelin sheath and oligodendrocytes, resulting in demyelination in the central nervous system. A well-established animal model of multiple sclerosis, experimental autoimmune encephalomyelitis (EAE), was used to examine the effect of the subject alkyl halides on this autoimmune and inflammatory condition of the central nervous system. Lewis rats were given intradermal footpad injections of 50 μg guinea pig myelin basic protein (MBP) and complete Freund's adjuvant (CFA) to induce experimental allergic encephalomyelitis (EAE). Daily oral administration of vehicle, methylene chloride or iodoform was started on day 8 post-inoculation and continued to the end of the study. Animals were monitored and scored on a 0 (normal) to 3 (complete hindlimb paralysis) scale. Daily mean±SD clinical scores are shown. As shown in FIG. 18, while the animals treated with MeCl show improvement over the controls, animals treated with iodoform did not display any clinical signs compared to controls.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A pharmaceutical composition for the treatment of vascular, inflammatory and immune disorders in a mammal, said pharmaceutical composition comprising at least one alkyl halide.

2. The pharmaceutical composition according to claim 1, wherein said alkyl halide is selected from the group consisting of haloalkanes and haloalkenes having from one to four carbon atoms and at least two halogen substitutions.

3. The pharmaceutical composition according to claim 1, wherein said alkyl halide comprises a dihalomethane.

4. The pharmaceutical composition according to claim 3, wherein said dihalomethane is methylene chloride.

5. The pharmaceutical composition according to claim 1, wherein said alkyl halide comprises a trihalomethane.

6. The pharmaceutical composition according to claim 5, wherein said trihalomethane is selected from the group consisting of iodoform and bromoform.

7. A method for extending the survival of an organ transplant in a recipient, said method comprising:
administering to said recipient a therapeutic amount of an alkyl halide, whereby the survival time of said organ transplant is extended.

8. A method for inhibiting the production of an inflammatory cytokine protein by cells capable of producing said inflammatory cytokine protein, said method comprising:
combining said cells with a therapeutic amount of an alkyl halide, whereby production of said inflammatory cytokine by said cells is inhibited.

9. A method for inhibiting an inflammatory response in a mammal, said method comprising:
contacting said mammal with a therapeutic amount of an alkyl halide, whereby said inflammatory response is inhibited.

10. The method according to claim 7, wherein said inflammatory response is associated with multiple sclerosis, septic shock, rheumatoid arthritis, Crohn's disease, colitis or ischemia/reperfusion injury.

11. A method for inhibiting vascular smooth muscle cell proliferation, said method comprising:
contacting said mammal with a therapeutic amount of an alkyl halide, whereby said VSMC proliferation is inhibited.

12. A method for inhibiting neointimal formation after vascular injury, said method comprising:
contacting said mammal with a therapeutic amount of an alkyl halide, whereby the formation of said neointima is inhibited.

13. The method according to any one of claims 7 to 12, wherein said alkyl halide is a haloform.

14. The method according to claim 13, wherein said haloform is selected from the group consisting of iodoform and bromoform.

15. The method according to any one of claims 7 to 14, wherein said therapeutic amount is sufficient to increase the blood carboxyhemoglobin level of said mammal to between about 1 and 10%.
